(19) United States
(12) Patent Application Publication
(10) Pub. No.: US 2011/0099665 A1
(43) Pub. Date: Apr. 28, 2011

(54) LEAFY COTYLEDON 1 TRANSCRIPTIONAL ACTIVATOR (LEC1) VARIANT POLYNUCLEOTIDES AND POLYPEPTIDES COMPOSITIONS AND METHODS OF INCREASING TRANSFORMATION EFFICIENCY

(75) Inventors: BO SHEN, JOHNSTON, IA (US); MITCHELL C. TARCZYNSKI, WEST DES MOINES, IA (US); IOWA STATE BANK, URBANDALE, IA (US); CHANGJIANG LI, BEIJING (CN); KIMBERLY F. GLASSMAN, ANKENY, IA (US); ARAGULA GURURAJ RAO, URBANDALE, IA (US); WILLIAM J. GORDON-KAMM, URBANDALE, IA (US)

(73) Assignee: PIONEER HI-BRED INTERNATIONAL, INC., JOHNSTON, IA (US)

(21) Appl. No.: 12/650,259

(22) Filed: Dec. 30, 2009

Related U.S. Application Data
(60) Provisional application No. 61/142,029, filed on Dec. 31, 2008.

(51) Int. Cl. A01H 5/00; C12N 15/82

(52) U.S. Cl. 800/278; 800/298; 435/468

(57) ABSTRACT

The present invention provides LEC1 variants. The LEC1 variants comprise a LEC1 A domain, a LEC1 B domain and a LEC1 C domain, where the LEC1 B domain has at least one mutation and/or is chimeric with respect to the LEC1 A or C domain. The invention also includes methods of preparing such LEC1 variants, and methods of using such LEC1 variants to modulate the level or activity of LEC1 variants in a plant cell. Modulation of LEC1 activity or levels can be used for different purposes such as increasing transformation efficiency, stimulating growth of somatic embryos, improving the growth and recovery of transformants, inducing apomixes, increasing transformation frequency, enhancing tissue culture response and the like.

### CROSS-REFERENCE TO RELATED APPLICATIONS

This application claims priority under 35 U.S.C. §119 of provisional application Ser. No. 61/142,029 filed Dec. 31, 2008, which application is hereby incorporated by reference in its entirety.

### BACKGROUND OF THE INVENTION

Major advances in plant transformation have occurred over the last few years. However, in major crop plants, such as maize and soybeans, serious genotype limitations still exist. Transformation of agronomically important maize inbred lines continues to be both difficult and time consuming. Traditionally, the only way to elicit a culture response has been by optimizing medium components and/or explant material and source. This has led to success in some genotypes, but most elite hybrids fail to produce a favorable culture response. While, transformation of model genotypes is efficient, the process of introgressing transgenes into production inbreds is laborious, expensive and time consuming. It would save considerable time and money if genes could be introduced into and evaluated directly in commercial hybrids.

Current methods for genetic engineering in maize require a specific cell type as the recipient of new DNA. These cells are found in relatively undifferentiated, rapidly growing callus cells or on the scutellar surface of the immature embryo (which gives rise to callus). Irrespective of the delivery method currently used, DNA is introduced into literally thousands of cells, yet transformants are recovered at frequencies of 10⁻⁵ relative to transiently-expressing cells. Exacerbating this problem, the trauma that accompanies DNA introduction directs recipient cells into cell cycle arrest and accumulating evidence suggests that many of these cells are directed into apoptosis or programmed cell death. (Bowen et al, Third International Congress of the International Society for Plant Molecular Biology, 1991, Abstract 1093). Therefore it would be desirable to provide improved methods capable of increasing transformation efficiency in a number of cell types.

Typically a selectable marker is used to recover transformed cells. Traditional selection schemes expose all cells to a phytotoxic agent and rely on the introduction of a resistance gene to recover transformants. Unfortunately, the presence of dying cells may reduce the efficiency of stable transformation. It would therefore be useful to provide a positive selection system for recovering transformants.

Further challenges exist in producing hybrid seeds. In particular, in hybrid crops, including grains, oil seeds, forages, fruits and vegetables, there are problems associated with the development and production of hybrid seeds. The process of cross-pollination of plants is laborious and expensive. In the cross-pollination process, the female plant must be prevented from being fertilized by its own pollen. Many methods have been developed over the years, such as detasseling in the case of corn, developing and maintaining male sterile lines, and developing plants that are incompatible with their own pollen, to name a few. Since hybrids do not breed true, the process must be repeated for the production of every hybrid seed lot.

To further complicate the process, inbred lines are crossed. For example in the case of corn, the inbreds can be low yielding. This provides a major challenge in the production of hybrid seed corn. In fact, certain hybrids cannot be commercialized at all due to the performance of the inbred lines. The production of hybrid seeds is consequently expensive, time consuming and provides known and unknown risks. It would therefore be valuable to develop new methods which contribute to the increase of production efficiency of hybrid seed.

As new traits are added to commercial crops by means of genetic engineering, challenges arise in “stacking” traits. In order to develop heritable stacked traits, the traits must be linked because of segregating populations. Improved methods for developing hybrid seed which would not require linking of the traits would significantly shorten the time for developing commercial hybrid seeds.

Gene silencing is another challenge in developing heritable traits with genetic engineering. Frequently gene silencing is seen following meiotic divisions. Elimination or reduction of this problem would advance the state of science and industry in this area. For these and other reasons, there is a need for the present invention.

### BRIEF SUMMARY OF THE INVENTION

Generally, it is an object of the present invention to provide variant polynucleotides and polypeptides of LEC1.

It is an object of the present invention to provide transgenic plants including the polynucleotides and polypeptides of the present invention.

Additionally, it is an object of the present invention to provide methods of modulating, in a plant cell or in a transgenic plant, the expression of the polynucleotides and polypeptides of the present invention.

A further still object of the present invention is to provide a method of increasing transformation efficiency.

A still further object of the present invention is to provide a method of increasing callus growth rate.

Therefore, in one aspect, the present invention relates to an isolated polynucleotide encoding a LEC1 variant polypeptide that includes a B domain of a first LEC1 protein and also includes LEC1 A and C domains where either the A or C domain or both are from a second LEC1 protein. In one aspect, the present invention relates to an isolated polynucleotide encoding a LEC1 variant polypeptide that includes a LEC1 A domain, a mutated LEC1 B domain, and a LEC1 C domain. In one aspect, the B domain of the LEC1 variant has less than 80% identity to the Arabidopsis LEC1 B domain of SEQ ID NO:1. In one aspect, the B domain of the LEC1 variant has an amino acid sequence of Met Pro Ile Ala Asn Val Ile (SEQ ID NO:14) and the sequence has at least one mutation. In another aspect, the present invention relates to an isolated LEC1 variant polynucleotide that encodes any of the polypeptides of SEQ ID NO:5, 7, 9, 11 or 13; a polynucleotide having any of the sequences of SEQ ID NO:4, 6, 8, 10 or 12; or a polynucleotide having at least 30 nucleotides in length which hybridizes under stringent conditions to any of the former polynucleotides. In another aspect, the present invention includes a polynucleotide having at least 90% sequence identity to any of the sequences of SEQ ID NO:4, 6, 8, 10 or 12. Provided herein in another aspect of the invention are isolated polynucleotides degenerate as a result of the genetic code for any of the LEC1 variants of the present invention. In another aspect, an isolated polynucleotide is complementary to a polynucleotide of any one of the LEC1 variants of the present invention.

The present invention also provides for an expression cassette having at least one polynucleotide encoding a LEC1 variant of the present invention. In another aspect, the present invention is directed to a host cell transfected with the recombinant expression cassette having a promoter functional in a plant operably linked to any of the isolated polynucleotides encoding polypeptides of the present invention.

In yet another aspect, the present invention relates to a transgenic plant including a recombinant expression cassette of a promoter functional in a plant operably linked to any of the isolated polynucleotides of the present invention. The present invention also provides for transgenic seed from the transgenic plant. In another aspect, the present invention is directed to a host cell transfected with the recombinant expression cassette of a promoter functional in a plant operably linked to any of the isolated polynucleotides of the present invention.

In one aspect, the present invention relates to an isolated LEC1 variant polypeptide that includes a LEC1 A domain, a mutated LEC1 B domain, and a LEC1 C domain. In one aspect, the present invention relates to an isolated LEC1 variant polypeptide that includes a LEC1 B domain from a first LEC1 protein and LEC1 A and C domains, where either the A or C domain or both are from a second LEC1 protein. In one aspect, the B domain of the LEC1 variant has less than 80% identity to the Arabidopsis B domain of SEQ ID NO:1. In another aspect, the B domain of the LEC1 variant includes an amino acid sequence of Met Pro Ile Ala Asn Val Ile (SEQ ID NO:14) and the sequence has at least one mutation In another aspect, the present invention relates to isolated LEC1 variant polypeptides of SEQ ID NO:5, 7, 9, 11 or 13; a polypeptide encoded by a polynucleotide having any of the sequences of SEQ ID NO:4, 6, 8, 10 or 12; and a polypeptide encoded by a nucleic acid molecule which hybridizes to any of the polynucleotides of SEQ ID NO:4, 6, 8, 10 or 12. In another aspect, the present invention includes a polypeptide that is at least 90% identical to the amino acid sequence of SEQ ID NO:5, 7, 9, 11 or 13 or a polypeptide encoded by a nucleic acid molecule that has a nucleotide sequence that is at least 90% identical to the sequences of SEQ ID NO:4, 6, 8, 10 or 12. In another aspect, the present invention relates to a LEC1 variant polypeptide that increases transformation efficiency.

In yet another aspect, the present invention relates to a transgenic plant of a recombinant expression cassette having a promoter functional in a plant operably linked to a polynucleotide encoding a LEC1 variant polypeptide of the present invention. The present invention also provides for transgenic seed from the transgenic plant. In another aspect, the present invention is directed to a host cell transfected with the recombinant expression cassette having a promoter functional in a plant operably linked to any of the polynucleotides encoding polypeptides of the present invention.

In a further aspect, the present invention relates to a method of modulating the level of LEC1 variant proteins in a plant cell. In one aspect, the method includes transforming a plant cell with a LEC1 variant polynucleotide operably linked to a promoter. The polynucleotide may be in sense or antisense orientation. The method further includes expressing the polynucleotide for an amount of time sufficient to modulate the LEC1 variant protein in the plant cell.

In another aspect, the present invention provides a method of modulating the level of LEC1 variant protein in a plant. The method includes stably transforming a plant cell with a LEC1 variant polynucleotide, in sense or antisense orientation, operably linked to a promoter functional in a plant cell. The method includes regenerating the transformed plant cell into a transformed plant that expresses the LEC1 variant polynucleotide in an amount sufficient to modulate the level of LEC1 variant protein in the plant.

In another aspect, the present invention relates to a method of increasing transformation efficiency. In one aspect, the method includes introducing a construct having a polynucleotide encoding a LEC1 variant of the present invention into a responsive plant cell under conditions sufficient to increase transformation efficiency.

Also provided are methods for increasing transformation efficiency, enhancing tissue culture response, inducing somatic embryogenesis, providing a method for positive selection, and/or producing somatic embryo by apomixis means.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

The invention can be more fully understood from the following detailed description and the accompanying drawing which form a part of this application.

FIG. 1 shows the comparison of various LEC1 B-domain amino acid sequences and their alignment.

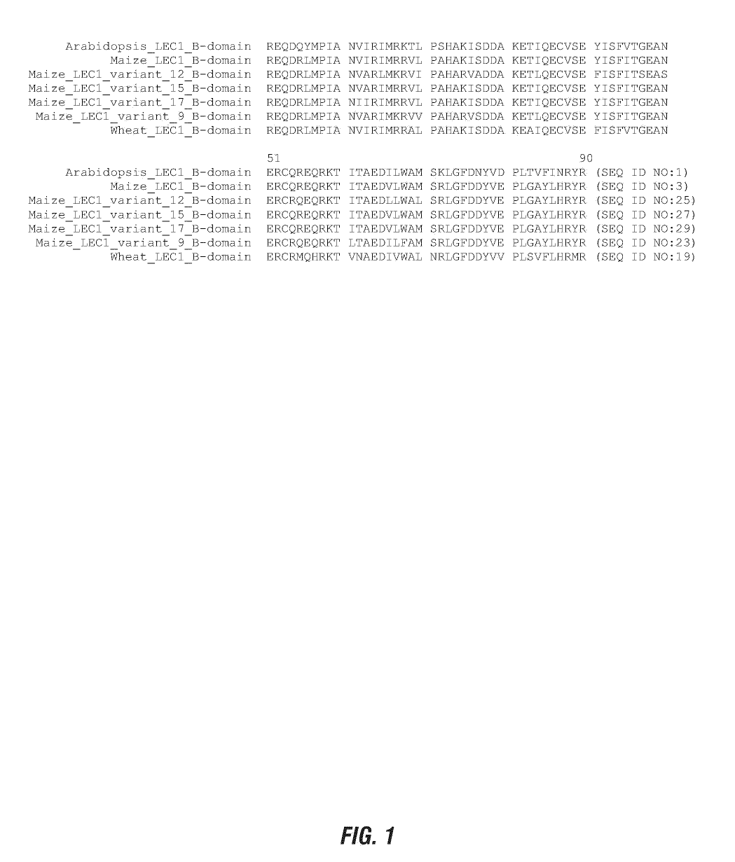

FIG. 2 shows a schematic representation of a maize LEC1 variant 9 expression cassette (PHP 26632) and a description of the features of the expression cassette.

FIG. 3 shows a schematic representation of a maize LEC1 variant 15 expression cassette (PHP 26810) and a description of the features of the expression cassette.

FIG. 4 shows a schematic representation of a wheat LEC1 expression cassette (PHP 25031) and a description of the features of the expression cassette.

FIG. 5 shows a schematic representation of a maize/wheat chimeric LEC1 expression cassette (PHP 26063) and a description of the features of the expression cassette.

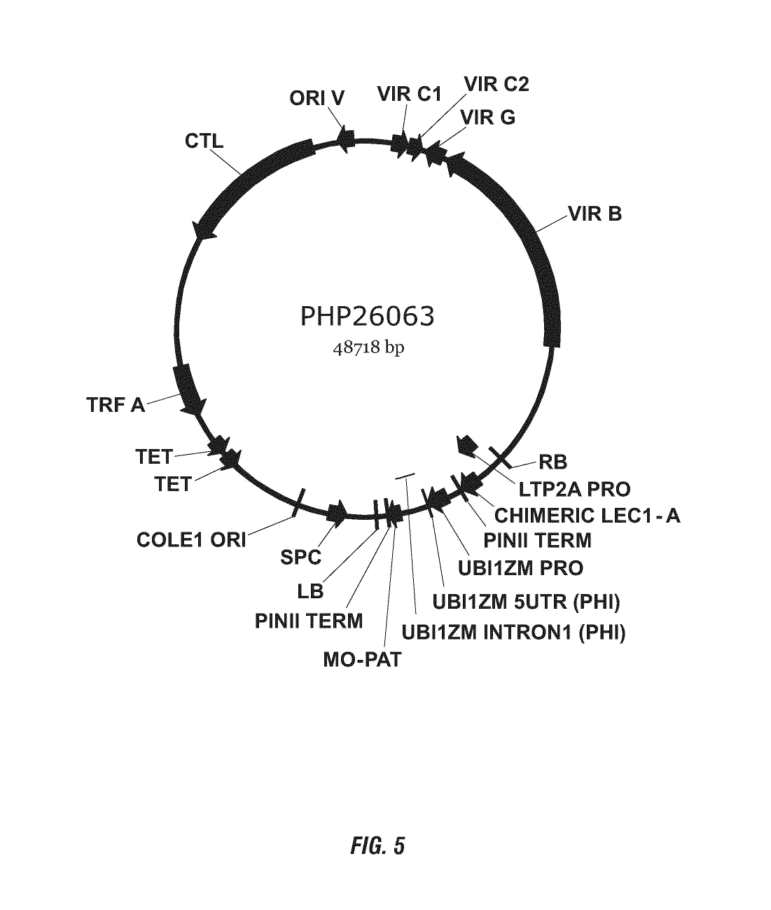

### BRIEF DESCRIPTION OF THE SEQUENCES

The application provides details of recombinant LEC1 sequences as shown in Table 1 below.

TABLE 1

| SEQ ID | Polynucleotide (pnt) | | |
|---|---|---|---|
| NO: | or polypeptide (ppt) | Length | Identification |
| 1 | ppt | 90 | Arabidopsis B domain |
| 2 | pnt | 270 | Maize LEC1 B domain |
| 3 | ppt | 90 | Maize LEC1 B domain |
| 4 | pnt | 837 | Maize LEC1 variant 9 (11 |
| | | | amino acid changes including 1 |
| | | | in signature sequence) |
| 5 | ppt | 278 | Maize LEC1 variant 9 (11 |
| | | | amino acid changes including 1 |
| | | | in signature sequence) |
| 6 | pnt | 837 | Maize LEC1 variant 12 (15 |
| | | | amino acid changes including 1 |
| | | | in signature sequence) |
| 7 | ppt | 278 | Maize LEC1 variant 12 (15 |
| | | | amino acid changes including 1 |
| | | | in signature sequence) |
| 8 | pnt | 837 | Maize LEC1 variant 15 (1 |
| | | | amino acid change in signature |
| | | | sequence) |
| 9 | ppt | 278 | Maize LEC1 variant 15 (1 |
| | | | amino acid change in signature |
| | | | sequence) |
| 10 | pnt | 837 | Maize LEC1 variant 17 (1 |
| | | | amino acid change in signature |
| | | | sequence) |
| 11 | ppt | 278 | Maize LEC1 variant 17 (1 |
| | | | amino acid change in signature |
| | | | sequence) |
| 12 | pnt | 837 | Maize chimeric LEC1 (maize |
| | | | A-wheat B-maize C) |
| 13 | ppt | 278 | Maize chimeric LEC1 (maize |
| | | | A-wheat B-maize C) |
| 14 | ppt | 7 | signature sequence of |
| | | | Arabidopsis B domain |
| 15 | ppt | 208 | Full length Arabidopsis LEC1 |
| 16 | pnt | 837 | Full length maize LEC1 |
| 17 | ppt | 278 | Full length maize LEC1 |
| 18 | pnt | 270 | wheat LEC1 B domain |
| 19 | ppt | 90 | wheat LEC1 B domain |
| 20 | pnt | 843 | Full length wheat LEC1 |
| 21 | ppt | 280 | Full length wheat LEC1 |
| 22 | pnt | 270 | B domain of Maize LEC1 |
| | | | variant 9 |
| 23 | ppt | 90 | B domain of Maize LEC1 |
| | | | variant 9 |
| 24 | pnt | 270 | B domain of Maize LEC1 |
| | | | variant 12 |
| 25 | ppt | 90 | B domain of Maize LEC1 |
| | | | variant 12 |
| 26 | pnt | 270 | B domain of Maize LEC1 |
| | | | variant 15 |
| 27 | ppt | 90 | B domain of Maize LEC1 |
| | | | variant 15 |
| 28 | pnt | 270 | B domain of Maize LEC1 |
| | | | variant 17 |
| 29 | ppt | 90 | B domain of Maize LEC1 |
| | | | variant 17 |

### DETAILED DESCRIPTION OF THE PREFERRED EMBODIMENT

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Leafy cotyledon1 (LEC1) is gene that has been has been identified a central regulator that plays multiple roles in embryogenesis. LEC1 was recently found to play a role in increasing the level of oil in the embryo of maize plants. See U.S. patent application Ser. No. 10/180,375, now U.S. Pat. No. 7,294,759, herein incorporated by reference in its entirety. LEC1 typically consists of primarily three domains: A, B, and C with the B domain reported as necessary for LEC1 activity in embryogenesis. Harada et al., Arabidopsis LEAFYT COTYLEDON1 Represents a Functionally Specialized Subunit of the CCAAT Binding Transcription Factor, P.N.A.S. (2003) 100(4): 2152-2156. The B domain typically includes about 90 residues and often has a conserved signature sequence of 7 residues of Met Pro Ile Ala Asn Val Ile (MPIANVI), (SEQ ID NO:14) sometimes referred to as the PIANO motif.

Experiments show that LEC1 variants having a mutated B domain or chimeric B domain produce oil content that is similar to that produced by endogenous wild type maize LEC1. See for example, Examples 3 and 4 described in provisional patent application No. 61/142,040 and patent application Ser. No. 12/650,188, incorporated by reference in its entirety. The successful coupling of the mutated LEC1 B domain or chimeric B domain with a LEC1 A and C domain to provide LEC1 activity is unexpected given that the B domain was previously described as critical for LEC1 function. Harada et al., Arabidopsis LEAFYT COTYLEDON1 Represents a Functionally Specialized Subunit of the CCAAT Binding Transcription Factor, P.N.A.S. (2003) 100(4): 2152-2156.

Accordingly, the present invention provides for LEC1 variants that are capable of increasing transformation efficiency of a plant. As used herein, the term “LEC1 variant” includes but is not limited to the sequences disclosed herein, such as chimeric LEC1 sequences and LEC1 sequences having a mutated B domain, their conservatively modified variants, regardless of source and any other variants which retain the biological properties of the LEC1, for example, LEC1 activity as disclosed herein.

In some examples, LEC1 variant polynucleotides of the invention can have at least about 50%, 55%, 60%, 65%, 70%, 75%, 80%, 85%, 90%, 91%, 92%, 93%, 95%, 95%, 96%, 97%, 98%, 99% or more sequence identity to any of the polynucleotides of SEQ ID NOS: 4, 6, 8, 10 or 12 and are encompassed by the invention. Also included are isolated polynucleotides that encode polypeptides having at least about 50%, 55%, 60%, 65%, 70%, 75%, 80%, 85%, 90%, 91%, 92%, 93%, 95%, 95%, 96%, 97%, 98%, 99% or more sequence identity to any of the polypeptides of SEQ ID NO: 5, 7, 9, 11 or 13. Sequence alignment programs and parameters described elsewhere herein can be used to determine sequence identity to that particular polynucleotide.

As used herein, the term “chimeric LEC1” refers to a LEC1 polynucleotide or polypeptide sequence containing a B domain from one LEC1 sequence and a nucleotide or amino acid sequence of a LEC1 A and/or C domain of an additional LEC1, for example, from a different plant. The term “LEC1 variant polypeptide” refers to one or more amino acid sequences. The term is also inclusive of fragments, homologs, alleles or precursors (e.g., preproproteins or proproteins) thereof. A “LEC1 variant protein” comprises a LEC1 variant polypeptide. In some examples, LEC1 variant polypeptides of the invention can have at least about 50%, 55%, 60%, 65%, 70%, 75%, 80%, 85%, 90%, 91%, 92%, 93%, 95%, 95%, 96%, 97%, 98%, 99% or more sequence identity to any of the polypeptides of SEQ ID NOS: 5, 7, 9, 11 or 13 and are encompassed by the invention. Sequence alignment programs and parameters described elsewhere herein can be used to determine sequence identity to that particular polypeptide. Unless otherwise stated, the term “LEC1 variant nucleic acid” means a nucleic acid comprising a polynucleotide (“LEC1 variant polynucleotide”) encoding a LEC1 variant polypeptide.

As used interchangeably herein, a “LEC1 activity”, “biological activity of LEC1” or “functional activity of LEC1”, refers to an activity exerted by a LEC1 protein, polypeptide or portion thereof as determined in vivo, or in vitro, according to standard techniques.

In one aspect, maintained or maintaining LEC1 activity is at least one or more of the following activities either in vivo or in vitro: (i) maintaining transformation efficiency of a plant, (ii) maintaining the growth rate of transformants, e.g. callus, (iii) altering embryo development, (iv) maintaining transformation frequency, (v) improving the embryonic character of calli, (vi) stimulating growth of somatic embryos, (vii) initiating formation of embryo-like structures, (viii) inducing apomixes, (ix) inducing somatic embryogenesis, (x) enhancing tissue culture response, (xi) any of the activities of (i) to (x).

In one aspect, increased or increasing LEC1 activity is at least one or more of the following activities either in vivo or in vitro: (i) increasing transformation efficiency of a plant, (ii) increasing the growth rate of transformants, e.g. callus, (iii) altering embryo development, (iv) increasing transformation frequency, (v) improving the embryonic character of calli, (vi) stimulating growth of somatic embryos, (vii) initiating formation of embryo-like structures, (viii) increasing the recovery of transformants, (ix) inducing apomixes, (x) inducing somatic embryogenesis, (xi) enhancing tissue culture response, (xii) increasing the recovery of regenerated plants, (xiii) any of the activities of (i) to (xii). Plants which can be used in the method of the invention include monocotyledonous and dicotyledonous plants.

Transformation efficiency may be increased in plants having LEC1 variants of the present invention relative to the transformation efficiency of a control plant that is non-transgenic for a LEC1 variant of the present invention. Transformation may be carried out by any suitable method, including but not limited to, Agrobacterium or particle bombardment-mediated transformation. For example, increased transformation efficiency of a plant may be assessed by comparing transformation frequency of transgenic plants and non-transgenic control plants. See, for example, See, U.S. Pat. No. 6,825,397. Preferably, the transformation efficiency in a plant transgenic for a LEC1 variant (or transformed plant cell, plant component, plant tissue, or plant organ) of the invention is at least 0.5%, 1%, 2%, 3%, 4%, 5%, 6%, 7%, 8%, 9%, 10%, 11%, 12%, 13%, 14%, 15% or 20%, 25%, 30%, 35%, 40% and preferably 45% or 50% greater than the transformation efficiency exhibited in a control plant that is non-transgenic with respect to the LEC1 variant (or control plant cell, plant component, plant tissue, or plant organ). In other preferred embodiments, the level of transformation efficiency is 50% greater, 60% greater, and more preferably even 75% or 90% greater than a control. The frequency of transformation efficiency is measured by conventional methods. See, U.S. Pat. No. 6,825,397. The transformation efficiency by the LEC1 variant may be observed by monitoring the effect on the host cell. The effect of the LEC1 variant may be monitored by any suitable means as known to one skilled in the art and as described herein.

The present invention relates to LEC1 variant polynucleotides and polypeptides and fragments thereof. As described herein, the inventors have identified novel LEC1 variants that have a LEC1 B domain that is mutated or is chimeric with respect to the LEC1 A or C domain. Any LEC1 B domain that has one or more mutations may be employed in the present invention, including any mutation that increases, enhances or otherwise maintains the effectiveness of the corresponding gene product (protein) so that the LEC1 variant is functional in performing any of its LEC1 activities.

As appreciated by one ordinarily skilled in the art, it is possible to utilize a LEC1 B domain polynucleotide sequence from any number of plants, including but not limited to an Arabidopsis, maize, soybean, sunflower, sorghum, canola, wheat, alfalfa, cotton, rice, barley, millet, Brassica, or coconut plant. One exemplary sequence of LEC1 B domains includes but is not limited to GenBank Accession No. AY264284.

The mutations in the LEC1 B domains employed in the present invention may be induced or naturally occurring. Mutations may be generated by any number of suitable techniques and approaches, including, for example, chemical induction of mutations, for example, by chemically treating LEC1 DNA with ultraviolet irradiation to induce mutations or through genetic engineering using recombinant DNA techniques. See also Example 3, described herein.

LEC1 variants may include a B domain that has all seven residues of the PIANO motif, that is the sequence of Met Pro Ile Ala Asn Val Ile (also referred to as MPIANVI) (SEQ ID NO:14). In other embodiments, the LEC1 variants may include a B domain that is mutated in the MPIANVI sequence (SEQ ID NO:14). Exemplary mutations of the MPIANVI sequence of the B domain include without limitation a substitution of the isoleucine at position 13 of SEQ ID NO:3 with an alanine or a substitution of the valine at position 12 of SEQ ID NO:3 with an isoleucine. The LEC1 variants may include B domains that have mutations that lie outside of the MPIANVI sequence (SEQ ID NO:14). Other combinations of mutations within and outside of the MPIANVI sequence (SEQ ID NO:14) may also be constructed and utilized. In one aspect, the B domain has less than 80% identity to the Arabidopsis LEC1 B domain of SEQ ID NO:1. In one aspect, the B domain has the amino acid sequence of Met Pro Ile Ala Asn Val Ile (SEQ ID NO:14).

In one aspect, the B domain of the LEC1 variant polynucleotide includes a mutation that includes one or more nucleotide substitutions at one or more of positions of 34, 36, 37, 38, 43, 45, 49, 50, 58, 73, 74, 75, 76, 78, 79, 81, 100, 102, 122, 139, 141, 149, 161, 162, 164, 181, 183, 196, 198, 203, 204 or 208 of the polynucleotide of SEQ ID NO:2 that encodes the maize LEC1 B domain. In one aspect, the B domain of the LEC1 variant polynucleotide includes a mutation that results in one or more nucleotide substitutions, for example, a guanosine substitution for adenosine at position 34, a guanosine substitution for cytidine at position 36, a guanosine substitution for adenosine at position 37, a cytidine substitution for thymidine at position 38, a cytidine substitution for adenosine at position 43, a guanosine substitution for cytidine at position 45, an adenosine substitution for cytidine at position 49, an adenosine substitution for guanosine at position 50, an adenosine or a guanosine substitution for cytidine at position 58, a cytidine substitution for adenosine at position 73, a guanosine substitution for adenosine at position 74, a cytidine substitution for guanosine at position 75, a guanosine substitution for adenosine at position 76, a guanosine substitution for cytidine at position 78, a guanosine substitution for thymidine at position 79, a cytidine substitution for guanosine at position 81, a cytidine substitution for adenosine at position 100, a guanosine substitution for cytidine at position 102, a thymidine substitution for adenosine at position 122, an adenosine substitution for guanosine at position 139, a cytidine substitution for guanosine at position 141, a guanosine substitution for adenosine at position 149, a guanosine substitution for adenosine at position 161, a cytidine substitution for guanosine at position 162, an adenosine substitution for guanosine at position 164, a cytidine substitution for adenosine at position 181, a guanosine substitution for cytidine at position 183, a cytidine or adenosine substitution for guanosine at position 196, a cytidine substitution for guanosine at position 198, a thymidine substitution for guanosine at position 203, a cytidine substitution for guanosine at position 204, a cytidine substitution for adenosine at position 208 of SEQ ID NO:2 or a combination thereof.

In one aspect, the B domain of the LEC1 variant polypeptide includes a mutation that includes a mutation that is an amino acid substitution at one or more of positions 15, 17, 20, 25, 26, 27, 34, 41, 47, 50, 54, 55, 61, 65, 66, 67, or 70 of the maize LEC1 B domain of the polypeptide of SEQ ID NO:3. In one aspect, the B domain of the LEC1 variant polypeptide includes a mutation that results in at least one mutation which is a substitution of the isoleucine at position 15 of SEQ ID NO:3 with a leucine, a substitution of the arginine position 17 of SEQ ID NO:3 with a lysine, a substitution of the leucine at position 20 of SEQ ID NO:3 with a valine or an isoleucine, a substitution of the lysine at position 25 of SEQ ID NO:3 with an arginine, a substitution of the isoleucine at position 26 of SEQ ID NO:3 with a valine, a substitution of the serine at position 27 of SEQ ID NO:3 with an alanine, a substitution of the isoleucine at position 34 of SEQ ID NO:3 with a leucine, a substitution of the tyrosine at position 41 of SEQ ID NO:3 with a phenylalanine, a substitution of the glycine at position 47 of SEQ ID NO:3 with a serine, a substitution of the asparagine at position 50 of SEQ ID NO:3 with a serine, a substitution of the glutamine at position 54 of SEQ ID NO:3 with an arginine, a substitution of the arginine at position 55 of SEQ ID NO:3 with a glutamine, a substitution of the isoleucine at position 61 of SEQ ID NO:3 with a leucine, a substitution of the aspartic acid at position 65 of SEQ ID NO:3 with an isoleucine, a substitution of the valine at position 66 of SEQ ID NO:3 with a leucine, a substitution of the tryptophan at position 67 of SEQ ID NO:3 with a phenylalanine; or a substitution of the methionine at position 70 of SEQ ID NO:3 with an leucine or a combination thereof. The mutated B domain sequence may be flanked by LEC1 A and C domains from the same or different plants as the B domain. Exemplary sequences of LEC1 A and C domains are known and include but are not limited to GenBank Accession No. AY264284.

Any number of different A, B and C domains of LEC1 may be joined together and are included in the present invention so long as the resulting chimeric LEC1 variant comprises an A, B and C domain where the B domain is of a different plant than either the A or C domain and the resulting LEC1 sequence has LEC1 activity. For example, in one aspect, the invention features a LEC1 variant that has a maize LEC1 A domain, a wheat LEC1 B domain and a maize LEC1 C domain. In one aspect, it is possible to utilize LEC1 A, B and C domain polynucleotide sequences from any number of plants, including, for example, Arabidopsis, maize, soybean, sunflower, sorghum, canola, wheat, alfalfa, cotton, rice, barley, millet, Brassica, or coconut plants. One exemplary sequence of LEC1 B domains includes but is not limited to GenBank Accession No. AY264284. Such a sequence may be incorporated into a nucleic acid sequence which encodes a chimeric LEC1 having the B domain inserted in frame between the LEC1 A and C domains. Exemplary sequences of LEC1 A and C domains include but are not limited to those found in GenBank Accession No. AY264284.

Thus, different LEC1 variants, including mutated or chimeric B domains, can be screened for those which retain LEC1 activity in the context of LEC1 A and C domains. As appreciated by those skilled in the art, LEC1 activity may be determined in any number of ways. For example, LEC1 activity of LEC1 variants of the present invention may be determined using complementation studies, e.g. the ability of a LEC1 variant to complement an Arabidopsis lec1 mutant and produce viable plants. See, for example, Example 3 as described herein. Other suitable methods include preparing a nucleic acid encoding a LEC1 variant and inserting the LEC1 variant sequence into an expression vector capable of expressing that sequence in a host plant cell, transforming a suitable host cell with the vector, and assaying for the transformation efficiency compared to the transformation efficiency from an appropriate control.

The present invention provides novel sequences and methods for increasing transformation efficiency of a plant cell. In particular, the polynucleotides and polypeptides of the present invention can be used to generate transgenic plants expressing LEC1 variants of the present invention. Uses of such LEC1 variants and fragments include increasing transformation efficiency of a plant. LEC1 variants are of interest, in part, because they allow for functional aspects of LEC1, for example, transformation efficiency of a plant cell, while providing alternative polynucleotides to combat gene silencing. Advantageously, plants transgenic for LEC1 variants of the present invention may be easily identified as the sequences differ in nucleotide and amino acid sequences from endogenous wild type LEC1.

Modulation of the LEC1 variants of the present invention would provide a mechanism for manipulating a plant's transformation efficiency. Accordingly, the present invention provides methods, polynucleotides, and polypeptides for the production of plants with maintained or increased transformation efficiency. In one aspect, the methods include introducing into a plant cell, plant tissue or plant one or more polynucleotides encoding LEC1 variant polypeptides having LEC1 activity. This may be accomplished by introducing the LEC1 variant polynucleotides driven by any number of promoters, for example, a constitutive promoter, such as a ubiquitin promoter, a seed-preferred promoter such as EAP1, a Ltp (lipid transfer protein) promoter, namely the Ltp2 gene promoter, into the plant nuclear genome. Exemplary promoters suitable for expression of the LEC1 variants will be appreciated by those skilled in the art and specific examples are described elsewhere herein.

The expression level of the chimeric LEC1 polypeptide may be measured directly, for example, by measuring the level of the chimeric LEC1 polypeptide in the plant by Western, or indirectly, for example, by measuring the LEC1 activity of the LEC1 polypeptide in the plant. Methods for determining the LEC1 activity may be determined using standard techniques such as NMR or GC. LEC1 activity may also include evaluation of phenotypic changes, such as increased transformation efficiency in a plant. Examples of phenotypic changes include but are not limited to increased transformation efficiency.

Increased transformation efficiency may be achieved through LEC1 variants of the present invention. Thus, modulation of activity of the LEC1 variants of the present invention in a plant cell provides a novel strategy for increasing transformation efficiency of a plant cell. Accordingly, the present invention further provides plants or plant cells having altered transformation efficiency, tissue culture response, somatic embryo-genesis, and transformation frequency.

A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; or (d) a plant or plant cell of the same genotype as the starting material but which has been transformed with a construct expressing maize LEC1.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5^{th} ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

### DEFINITIONS

In describing the present invention, the following terms will be employed, and are intended to be defined as indicated below.

For the purposes of the present invention, “grain”, “seed”, and “kernel”, will be used interchangeably.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Yamao, et al., (1985) Proc. Natl. Acad. Sci. USA 82:2306-9), or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the invention, which contains a vector and supports the replication and/or expression of the expression vector. Host cells may be prokaryotic cells such as E. coli, or eukaryotic cells such as yeast, insect, plant, amphibian, or mammalian cells. Preferably, host cells are monocotyledonous or dicotyledonous plant cells, including but not limited to maize, sorghum, sunflower, soybean, wheat, alfalfa, rice, cotton, canola, barley, millet, and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “LEC1 variant nucleic acid” means a nucleic acid comprising a polynucleotide (“LEC1 variant polynucleotide”) encoding a full length or partial length LEC1 variant polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum. A particularly preferred plant is Zea mays.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibres, xylem vessels, tracheids, or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated, and inducible promoters constitute the class of “non-constitutive” promoters. A “constitutive” promoter is a promoter, which is active under most environmental conditions, for example, the ubiquitin gene promoter UBI (GenBank accession no S94464).

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention; or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, N.Y. (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity,” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (TFASTA and FASTA) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90%, and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

### Nucleic Acids

The present invention provides, inter alia, isolated nucleic acids of RNA, DNA, and analogs and/or chimeras thereof, comprising a LEC1 variant polynucleotide.

The present invention also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 4 of Murray, et al., supra. Codon optimization methods may also be found in patent application publication no. 20060236424.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G> 7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395); or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT publication No. 96/19256. See also, Zhang, et al., (1997) Proc. Natl. Acad. Sci. USA 94:4504-9; and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used in the practice of the invention, including the native promoter of an endogenous LEC1 polynucleotide sequence of the crop plant of interest. The promoters can be selected based on the desired outcome. The nucleic acids can be combined with constitutive, tissue-preferred, inducible, or other promoters for expression in plants.

A plant promoter or promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30); and maize H3 histone (Lepetit, et al., (1992) Mol. Gen. Genet. 231:276-85; and Atanassvoa, et al., (1992) Plant Journal 2(3):291-300); ALS promoter, as described in PCT Application No. WO 96/30530; and other transcription initiation regions from various plant genes known to those of skill. For the present invention, ubiquitin is the preferred promoter for expression in monocot plants.

Tissue-preferred promoters can be utilized to target enhanced LEC1 expression within a particular plant tissue. By “tissue-preferred” is intended to mean that expression is predominately in a particular tissue, albeit not necessarily exclusively in that tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 255(3):337-353; Russell et al. (1997) Transgenic Res. 6(2):157-168; Rinehart et al. (1996) Plant Physiol. 112(3):1331-1351; Van Camp et al. (1996) Plant Physiol. 112(2):525-535; Canevascini et al. (1996) Plant Physiol. 112(2):513-525; Yamamoto et al. (1995) Plant Cell Physiol. 35(5):773-778; Lam (1995) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 5(3):595-505. Such promoters can be modified, if necessary, for weak expression. See, also, U.S. Patent Application No. 2003/0074698, herein incorporated by reference.

Embryonic-preferred promoters can be utilized for expression of LEC1 within the embryo. Embryonic-preferred promoters include but are not limited to Oleosin promoter, EAP1 promoter, or Ltp2 promoter.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes, or alternatively from another plant gene, or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50; and An, et al., (1989) Plant Cell 1:115-22); and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, N.Y. (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119, and hereby incorporated by reference), or signal peptides which target proteins to the plastids such as that of rapeseed enoyl-Acp reductase (Verwaert, et al., (1994) Plant Mol. Biol. 26:189-202) are useful in the invention.

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al. (1987), Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11, and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation, and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7^{th} ed., 1992).

Appropriate vectors for expressing proteins of the present invention in insect cells are usually derived from the SF9 baculovirus. Suitable insect cell lines include mosquito larvae, silkworm, armyworm, moth, and Drosophila cell lines such as a Schneider cell line (see, e.g., Schneider, (1987) J. Embryol. Exp. Morphol. 27:353-65).

As with yeast, when higher animal or plant host cells are employed, polyadenylation or transcription terminator sequences are typically incorporated into the vector. An example of a terminator sequence is the polyadenylation sequence from the bovine growth hormone gene. Other useful terminators for practicing this invention include, but are not limited to, pinII (See An et al. (1989) Plant Cell 1(1):115-122), glb1 (See Genbank Accession #L22345), gz (See gzw64a terminator, Genbank Accession #S78780), and the nos terminator from Agrobacterium.

Sequences for accurate splicing of the transcript may also be included. An example of a splicing sequence is the VP1 intron from SV40 (Sprague et al., J. Virol. 45:773-81 (1983)). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the LEC1 variant polynucleotide placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert LEC1 variant polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119. In one aspect, the expression cassette includes the “monocot-optimized” PAT gene (moPAT) driven by the ubiquitin promoter. See, for example, U.S. Pat. No. 6,096,947.

The isolated polynucleotides or polypeptides may be introduced into the plant by one or more techniques typically used for direct delivery into cells. Such protocols may vary depending on the type of organism, cell, plant or plant cell, i.e. monocot or dicot, targeted for gene modification. Suitable methods of transforming plant cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. O. L. Gamborg & G. C. Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren & Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N Y (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); L1, et al., (1993) Plant Cell Reports 12:250-255; and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. Pat. No. 913,914, filed Oct. 1, 1986, as referenced in U.S. Pat. No. 5,262,306, issued Nov. 16, 1993; and Simpson, et al., (1986) Plant Mol. Biol. 6:403-15 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application No. 604 662 A1 discloses a method for transforming monocots using Agrobacterium. European Application No. 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. Pat. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731; and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

Increasing the Activity and/or Level of a LEC1 Variant Polypeptide

Methods are provided to increase the activity and/or level of the LEC1 variant polypeptide of the invention. An increase in the level and/or activity of the LEC1 variant polypeptide of the invention can be achieved by providing to the plant a LEC1 variant polypeptide. The LEC1 variant polypeptide can be provided by introducing the amino acid sequence encoding the LEC1 variant polypeptide into the plant, introducing into the plant a nucleotide sequence encoding a LEC1 variant polypeptide or alternatively by modifying a genomic locus encoding the LEC1 variant polypeptide of the invention.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having enhanced nitrogen utilization activity. It is also recognized that the methods of the invention may employ a polynucleotide that is not capable of directing, in the transformed plant, the expression of a protein or an RNA. Thus, the level and/or activity of a LEC1 variant polypeptide may be increased by altering the gene encoding the LEC1 variant polypeptide or its promoter. See, e.g., Kmiec, U.S. Pat. No. 5,565,350; Zarling, et al., PCT/US93/03868. Therefore mutagenized plants that carry mutations in LEC1 variant genes, where the mutations increase expression of the LEC1 variant gene or increase the LEC1 variant activity of the encoded LEC1 variant polypeptide are provided.

Reducing the Activity and/or Level of a LEC1 Variant Polypeptide

Reducing the Activity and/or Level of a LEC1 Variant Polypeptide

Methods are provided to reduce or eliminate the activity of a LEC1 variant polypeptide of the invention by transforming a plant cell with an expression cassette that expresses a polynucleotide that inhibits the expression of the LEC1 variant polypeptide. The polynucleotide may inhibit the expression of the LEC1 variant polypeptide directly, by preventing transcription or translation of the LEC1 variant messenger RNA, or indirectly, by encoding a polypeptide that inhibits the transcription or translation of a LEC1 variant gene encoding LEC1 variant polypeptide. Methods for inhibiting or eliminating the expression of a gene in a plant are well known in the art, and any such method may be used in the present invention to inhibit the expression of LEC1 variant polypeptide.

In accordance with the present invention, the expression of LEC1 variant polypeptide is inhibited if the protein level of the LEC1 variant polypeptide is less than 70% of the protein level of the same LEC1 variant polypeptide in a plant that has not been genetically modified or mutagenized to inhibit the expression of that LEC1 variant polypeptide. In particular embodiments of the invention, the protein level of the LEC1 variant polypeptide in a modified plant according to the invention is less than 60%, less than 50%, less than 40%, less than 30%, less than 20%, less than 10%, less than 5%, or less than 2% of the protein level of the same LEC1 variant polypeptide in a plant that is not a mutant or that has not been genetically modified to inhibit the expression of that LEC1 variant polypeptide. The expression level of the LEC1 variant polypeptide may be measured directly, for example, by assaying for the level of LEC1 variant polypeptide expressed in the plant cell or plant, or indirectly, for example, by measuring the activity of the LEC1 variant polypeptide in the plant cell or plant, or by measuring the phenotypic changes in the plant. Methods for performing such assays are described elsewhere herein.

In other embodiments of the invention, the activity of the LEC1 variant polypeptides is reduced or eliminated by transforming a plant cell with an expression cassette comprising a polynucleotide encoding a polypeptide that inhibits the activity of a LEC1 variant polypeptide. The enhanced LEC1 activity of a LEC1 variant polypeptide is inhibited according to the present invention if the LEC1 variant activity of the LEC1 variant polypeptide is less than 70% of the LEC1 variant activity of the same LEC1 variant polypeptide in a plant that has not been modified to inhibit the LEC1 variant activity of that LEC1 variant polypeptide. In particular embodiments of the invention, the LEC1 variant activity of the LEC1 variant polypeptide in a modified plant according to the invention is less than 60%, less than 50%, less than 40%, less than 30%, less than 20%, less than 10%, or less than 5% of the LEC1 variant activity of the same LEC1 variant polypeptide in a plant that that has not been modified to inhibit the expression of that LEC1 variant polypeptide. The LEC1 variant activity of a LEC1 variant polypeptide is “eliminated” according to the invention when it is not detectable by the assay methods described elsewhere herein. Methods of determining the alteration of LEC1 activity of a LEC1 variant polypeptide are described elsewhere herein.

In other embodiments, the activity of a LEC1 variant polypeptide may be reduced or eliminated by disrupting the gene encoding the LEC1 variant polypeptide. The invention encompasses mutagenized plants that carry mutations in LEC1 variant genes, where the mutations reduce expression of the LEC1 variant gene or inhibit the LEC1 activity of the encoded LEC1 variant polypeptide.

Thus, many methods may be used to reduce or eliminate the activity of a LEC1 variant polypeptide. In addition, more than one method may be used to reduce the activity of a single LEC1 variant polypeptide.

In some embodiments of the present invention, a plant is transformed with an expression cassette that is capable of expressing a polynucleotide that inhibits the expression of a LEC1 variant polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one LEC1 variant polypeptide is an expression cassette capable of producing an RNA molecule that inhibits the transcription and/or translation of at least one LEC1 variant polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Compositions of the invention comprise sequences encoding maize seed proteins and variants and fragments thereof. Methods of the invention involve the use of, but are not limited to, transgenic expression, antisense suppression, co-suppression, RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING, and biosynthetic competition to manipulate, in plants and plant seeds and grains, the expression of seed proteins, including, but not limited to, those encoded by the sequences disclosed herein.

Other methods for decreasing or eliminating the expression of genes include the transgenic application of transcription factors (Pabo, C. O., et al. (2001) Annu Rev Biochem 70, 313-40; and Reynolds, L., et al (2003), Proc Natl Acad Sci USA 100, 1615-20.), and homologous recombination methods for gene targeting (see U.S. Pat. No. 6,187,994).

Similarly, it is possible to eliminate the expression of a single gene by replacing its coding sequence with the coding sequence of a second gene using homologous recombination technologies (see Bolon, B. Basic Clin. Pharmacol. Toxicol. 95:4, 12, 154-61 (2004); Matsuda and Alba, A., Methods Mol. Bio. 259:379-90 (2004); Forlino, et. al., J. Biol. Chem. 274:53, 37923-30 (1999)).

### Modulating LEC1 Activity

Methods for increasing the level and/or activity of LEC1 variant polypeptides in a plant are discussed elsewhere herein. Briefly, such methods comprise providing a LEC1 variant polypeptide of the invention to a plant and thereby increasing the level and/or activity of the LEC1 variant polypeptide. In other embodiments, a LEC1 variant nucleotide sequence encoding a LEC1 variant polypeptide can be provided by introducing into the plant a polynucleotide comprising a LEC1 variant nucleotide sequence of the invention, expressing the LEC1 variant sequence, thereby increasing the level and/or activity of the LEC1 variant polypeptide. In other embodiments, the LEC1 variant nucleotide construct introduced into the plant is stably incorporated into the genome of the plant.

As discussed above, one of skill will recognize the appropriate promoter to use to modulate the level/activity of a LEC1 variant in the plant. Exemplary promoters for this embodiment have been disclosed elsewhere herein.

In other embodiments, such plants have stably incorporated into their genome a nucleic acid molecule comprising a LEC1 variant nucleotide sequence of the invention operably linked to a promoter that drives expression in the plant cell.

### Producing Somatic Embryo by Apomixis Means.

Expression of a LEC1 variant polynucleotide may initiate formation of embryo-like structures and improve growth and recovery of transformants. LEC1 variant polynucleotides may induce apomixis. The term apomixis is used to describe asexual reproduction that replaces or substitutes sexual methods of reproduction. When apomixis occurs, embryos are produced from maternal tissue and use only the maternal genome. In many cases of apomixis maternal tissues such as the nucellus or inner integument “bud off” producing somatic embryos. These embryos then develop normally into seed. Since meiosis and fertilization are circumvented, the plants developing from such seed are genetically identical to the maternal plant. Expression of the LEC1 variant polynucleotide in the nucellus integument, or cell specific expression in the megaspore mother cell may trigger embryo formation from maternal tissues.

Producing a seed identical to the parent has many advantages. For example high yielding hybrids could be used in seed production to multiply identical copies of high yielding hybrid seed. This would greatly reduce seed cost as well as increase the number of genotypes which are commercially available. Genes can be evaluated directly in commercial hybrids since the progeny would not segregate. This would save years of back crossing. Apomixis would also provide a method of containment of transgenes when coupled with male sterility. The construction of male sterile autonomous agamospermy would prevent genetically engineered traits from hybridizing with weedy relatives. Apomixis would also facilitate gene stacking as hybrids could be successively re-transformed with various new traits and propagated via apomixis. The traits would not need to be linked since apomixis avoids the problems associated with segregation. Apomixis can also provide a reduction in gene silencing that is frequently seen following meiotic divisions. Since meiotic divisions never occur, it may be possible to eliminate or reduce the frequency of gene silencing. Apomixis can also be used stabilize desirable phenotypes with complex traits such as hybrid vigor. Such traits could easily be maintained and multiplied indefinitely via apomixis.

### Recalcitrant Tissues/Genotypes

Expression of the LEC1 variant polynucleotide may be used to stimulate embryo formation in tissues/genotypes normally not amenable to culture. Likewise it is an advantage to have ectopic expression in genotypes amenable to culture as this can increase the number of embryo precursor cells and/or increase the number that develop into embryos. This can lead to an increase in transformation frequency, increase the growth rate and embryogenic character of transgenic calli, reduce the time needed to recover regenerable calli, and make regeneration of vigorous fertile plants easier and more reproducible. Transient expression using RNA or protein may be sufficient to initiate the cascade of events leading to embryo formation. This would be valuable in such target tissues as maize scutella, immature leaf bases, etc. The LEC1 variant polynucleotide may also be used as a positive selectable marker, i.e. triggering embryogenesis in transgenic cells without killing the surrounding wild-type cells. The cells receiving the LEC1 variant polynucleotide would undergo embryogenesis or in tissues already undergoing embryogenesis expression of LEC1 variants would stimulate more rapid reiteration and growth of somatic embryos. Thus transformed cells can be selected by their more rapid development of embryos. Expression of the LEC1 variant polynucleotide in transformed cells may initiate embryo development and stimulate development of pre-existing embryos. Normally, LEC1 expression is necessary for proper embryo maturation in the latter stages of embryo development, and LEC1 variant transgene expression may also promote these processes. These combined effects on somatic embryogenesis may stimulate growth of transformed cells, as well as insure that transformed somatic embryos develop in a normal, viable fashion, increasing the capacity of transformed somatic embryos to germinate vigorously. Continued ectopic overexpression beyond embryo maturation may negatively impact germination and vegetative plant growth, which may necessitate down-regulation of the LEC1 transgene during these stages of development.

Expression of the LEC1 variant polynucleotide may stimulate growth in cells with the potential to initiate or maintain embryogenic growth. In addition, transformation methods that target certain reproductive tissues or cells, such as vacuum-infiltration of Agrobacterium into Arabidopsis, may have detrimental effects on recovery of transformants, e.g. triggering genes associated with embryogenesis. Expression of LEC1 variant polynucleotides in transformants may help improve transformant recovery.

Altering the Culture Medium to Suppress Somatic Embryogenesis in Non-Transformed Plant Cells and/or Tissues to Provide for a Positive Section Means of Transformed Plant Cells

Using the methods disclosed herein for controlling somatic embryogenesis, it may be possible to alter plant tissue culture media components to suppress somatic embryogenesis in a plant species of interest often having multiple components that potentially could be adjusted to impart this effect. Such conditions would not impart a negative or toxic in vitro environment for wild-type tissue, but instead would simply not produce a somatic embryogenic growth form. Introducing a transgene such as a LEC1 variant may stimulate somatic embryogenesis and growth in the transformed cells or tissue, providing a clear differential growth screen useful for identifying transformants.

Altering a wide variety of media components may modulate somatic embryogenesis by either stimulating or suppressing embryogenesis depending on the species and particular media component. Examples of media components which, when altered, can stimulate or suppress somatic embryogenesis include; the basal medium itself (macronutrient, micronutrients and vitamins; see T. A. Thorpe, 1981 for review, “Plant Tissue Culture: Methods and Applications in Agriculture”, Academic Press, NY), plant phytohormones such as auxins (indole acetic acid, indole butyric acid, 2,4-dichlorophenoxyacetic acid, naphthaleneacetic acid, picloram, dicamba and other functional analogues), cytokinins (zeatin, kinetin, benzyl amino purine, 2-isopentyl adenine and functionally-related compounds) abscisic acid, adenine, and gibberellic acid, and other compounds that exert “growth regulator” effects such as coconut water, casein hydrolysate, and proline, and the type and concentration of gelling agent, pH and sucrose concentration.

Changes in the individual components listed above (or in some cases combinations of components) have been demonstrated in the literature to modulate in vitro somatic embryogenesis across a wide range of dicotyledonous and monocotyledonous species. For a compilation of examples, see E. F. George et al. 1987. Plant Tissue Culture Media. Vol. 1: Formulations and Uses. Exergetics, Ltd., Publ., Edington, England.

### Method of Use for LEC1 Variant Polynucleotide, Expression Cassettes, and Additional Polynucleotides

The nucleotides, expression cassettes and methods disclosed herein are useful in varying the phenotype of a plant or plant cell. Various changes in phenotype are of interest including increasing transformation efficiency, stimulating growth of somatic embryos, improving the growth and recovery of transformants, inducing apomixis, increasing transformation frequency, improving the growth rate of callus, improving the embryonic character of calli, and enhancing tissue culture response and the like. These results can be achieved by providing expression of heterologous products in plants.

Genes of interest are reflective of the commercial markets and interests of those involved in the development of the crop. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

The polynucleotides of the present invention may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson, et al., (1987) Eur. J. Biochem. 165:99-106; and WO 98/20122); and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see U.S. Pat. No. 5,583,210), stalk strength, flowering time, or transformation technology traits such as cell cycle regulation or gene targeting (e.g., WO 99/61619; WO 00/17364; WO 99/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359; both of which are herein incorporated by reference); and rice (Musumura, et al., (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser, et al., (1986) Gene 48:109); and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

### Sequences:

Arabidopsis LEC1 Amino Acid Sequence with B-Domain

(SEQ ID NO: 1)
REQDQYMPIANVIRIMRKTLPSHAKISDDAKETIQECVSEYISFVTGE
ANERCQREQRKTITAEDILWAMSKLGFDNYVDPLTVFINRYR

### Maize LEC1 B Domain

(SEQ ID NO: 2)
cgcgagcaggaccggctgatgccgatcgcgaacgtgatccgcatcat
gcggcgcgtgctgccggcgcacgccaagatctcggacgacgccaagg
agacgatccaggagtgcgtgtcggagtacatcagcttcatcacgggg
gaggccaacgagcggtgccagcgggagcagcgcaagaccatcaccgc
cgaggacgtgctgtgggccatgagccgcctcggcttcgacgactacg
tcgagccgctcggcgcctacctccaccgctaccgc

### Maize LEC1 B Domain (Signature Amino Acids Underlined)

(SEQ ID NO: 3)
REQDRLMPIANVIRIMRRVLPAHAKISDDAKETIQECVSEYISFITGE
ANERCQREQRKTITAEDVLWAMSRLGFDDYVEPLGAYLHRYR

Maize LEC1 Variant 9 Nucleotide Sequence with B-Domain Underlined

(SEQ ID NO: 4)
atggactccagcagcttcctccctgccgccggcgcggagaatggctcg
gcggcgggcggcgccaacaatggcggcgctgctcagcagcatgcggcg
ccggcgatccgcgagcaggaccggctgatgccgatcgcgaacgtggcc
cgcatcatgaagcgcgtggtgccggcgcacgcccgcgtgtcggacgac
gccaaggagacgctgcaggagtgcgtgtcggagtacatcagccatgag
ccgcctcggcttcgacgactacgtcgagccgctcggcgcctacctcca
ccgctaccgcgagttcgagggcgacgcgcgcggcgtcgggctcgtccc
gggggccgccccatcgcgcggcggcgaccaccacccgcactccatgtc
gccagcggcgatgctcaagtcccgcgggccagtctccggagccgccat
gctaccgcaccaccaccaccaccacgacatgcagatgcacgccgccat
gtacgggggaacggccgtgcccccgccggccgggcctcctcaccacgg
cgggttcctcatgccacacccacagggtagtagccactacctgcctta
cgcgtacgagcccacgtacggcggtgagcacgccatggctgcatacta
tggaggcgccgcgtacgcgcccggcaacggcgggagcggcgacggcag
tggcagtggcggcggtggcgggagcgcgtcgcacacaccgcagggcag
cggcggcttggagcacccgcacccgttcgcgtacaagtag

### Maize LEC1 Variant 9 (11 Amino Acid Changes (Bold) Including 1 in Signature Sequence)

(SEQ ID NO: 5)
MDSSSFLPAAGAENGSAAGGANNGGAAQQHAAPAIREQDRLMPIANVA
RIMKRVVPAHARVSDDAKETLQECVSEYISFITGEANERCRQEQRKTL
TAEDILFAMSRLGFDDYVEPLGAYLHRYREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

Maize LEC1 Variant 12 Nucleotide Sequence with B-Domain Underlined

(SEQ ID NO: 6)
atggactccagcagcttcctccctgccgccggcgcggagaatggctcg
gcggcgggcggcgccaacaatggcggcgctgctcagcagcatgcggcg
ccggcgatccgcgagcaggaccggctgatgccgatcgcgaacgtggcc
cgcctgatgaagcgcgtgatcccggcgcacgcccgcgtggccgacgac
gccaaggagacgctgcaggagtgcgtgtcggagttcatcagcttcatc
acgagcgaggccagcgagcggtgccgccaggagcagcgcaagaccatc
accgccgaggacctcctgtgggccctgagccgcctcggcttcgacgac
tacgtcgagccgctcggcgcctacctccaccgctaccgcgagttcgag
ggcgacgcgcgcggcgtcgggctcgtcccgggggccgccccatcgcgc
ggcggcgaccaccacccgcactccatgtcgccagcggcgatgctcaag
tcccgcgggccagtctccggagccgccatgctaccgcaccaccaccac
caccacgacatgcagatgcacgccgccatgtacgggggaacggccgtg
cccccgccggccgggcctcctcaccacggcgggttcctcatgccacac
ccacagggtagtagccactacctgccttacgcgtacgagcccacgtac
ggcggtgagcacgccatggctgcatactatggaggcgccgcgtacgcg
cccggcaacggcgggagcggcgacggcagtggcagtggcggcggtggc
gggagcgcgtcgcacacaccgcagggcagcggcggcttggagcacccg
cacccgttcgcgtacaagtag

### Maize LEC1 Variant 12 (15 Amino Acid Changes (in Bold) Including 1 in Signature Sequence

(SEQ ID NO: 7)
MDSSSFLPAAGAENGSAAGGANNGGAAQQHAAPAIREQDRLMPIANVA
RLMKRVIPAHARVADDAKETLQECVSEFISFITSEASERCRQEQRKTI
TAEDLLWALSRLGFDDYVEPLGAYLHRYREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

Maize LEC1 Variant 15 Nucleotide Sequence with B-Domain Underlined

(SEQ ID NO: 8)
atggactccagcagcttcctccctgccgccggcgcggagaatggctcg
gcggcgggcggcgccaacaatggcggcgctgctcagcagcatgcggcg
ccggcgatccgcgagcaggaccggctgatgccgatcgcgaacgtggcc
cgcatcatgcggcgcgtgctgccggcgcacgccaagatctcggacgac
gccaaggagacgatccaggagtgcgtgtcggagtacatcagcttcatc
acgggggaggccaacgagcggtgccagcgggagcagcgcaagaccatc
accgccgaggacgtgctgtgggccatgagccgcctcggcttcgacgac
tacgtcgagccgctcggcgcctacctccaccgctaccgcgagttcgag
ggcgacgcgcgcggcgtcgggctcgtcccgggggccgccccatcgcgc
ggcggcgaccaccacccgcactccatgtcgccagcggcgatgctcaag
tcccgcgggccagtctccggagccgccatgctaccgcaccaccaccac
caccacgacatgcagatgcacgccgccatgtacgggggaacggccgtg
cccccgccggccgggcctcctcaccacggcgggttcctcatgccacac
ccacagggtagtagccactacctgccttacgcgtacgagcccacgtac
ggcggtgagcacgccatggctgcatactatggaggcgccgcgtacgcg
cccggcaacggcgggagcggcgacggcagtggcagtggcggcggtggc
gggagcgcgtcgcacacaccgcagggcagcggcggcttggagcacccg
cacccgttcgcgtacaagtag

### Maize LEC1 Variant 15 (1 Amino Acid Change in Signature Sequence in Bold, B Domain Underlined)

(SEQ ID NO: 9)
MDSSSFLPAAGAENGSAAGGANNGGAAQQHAAPAIREQDRLMPIANVA
RIMRRVLPAHAKISDDAKETIQECVSEYISFITGEANERCQREQRKTI
TAEDVLWAMSRLGFDDYVEPLGAYLHRYREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

Maize LEC1 Variant 17 Nucleotide Sequence with B-Domain Underlined

(SEQ ID NO: 10)
atggactccagcagcttcctccctgccgccggcgcggagaatggctcg
gcggcgggcggcgccaacaatggcggcgctgctcagcagcatgcggcg
ccggcgatccgcgagcaggaccggctgatgccgatcgcgaacatcatc
cgcatcatgcggcgcgtgctgccggcgcacgccaagatctcggacgac
gccaaggagacgatccaggagtgcgtgtcggagtacatcagcttcatc
acgggggaggccaacgagcggtgccagcgggagcagcgcaagaccatc
accgccgaggacgtgctgtgggccatgagccgcctcggcttcgacgac
tacgtcgagccgctcggcgcctacctccaccgctaccgcgagttcgag
ggcgacgcgcgcggcgtcgggctcgtcccgggggccgccccatcgcgc
ggcggcgaccaccacccgcactccatgtcgccagcggcgatgctcaag
tcccgcgggccagtctccggagccgccatgctaccgcaccaccaccac
caccacgacatgcagatgcacgccgccatgtacgggggaacggccgtg
cccccgccggccgggcctcctcaccacggcgggttcctcatgccacac
ccacagggtagtagccactacctgccttacgcgtacgagcccacgtac
ggcggtgagcacgccatggctgcatactatggaggcgccgcgtacgcg
cccggcaacggcgggagcggcgacggcagtggcagtggcggcggtggc
gggagcgcgtcgcacacaccgcagggcagcggcggcttggagcacccg
cacccgttcgcgtacaagtag

### Maize LEC1 Variant 17 (1 Amino Acid Change in Signature Sequence in Bold, B Domain Underlined)

(SEQ ID NO: 11)
MDSSSFLPAAGAENGSAAGGANNGGAAQQHAAPAIREQDRLMPIANII
RIMRRVLPAHAKISDDAKETIQECVSEYISFITGEANERCQREQRKTI
TAEDVLWAMSRLGFDDYVEPLGAYLHRYREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

### Maize Chimeric LEC1 Nucleotide Sequence (Maize A-Wheat B-Maize C, Wheat LEC1 B-Domain is Underlined)

(SEQ ID NO: 12)
atggactccagcagcttcctccctgccgccggcgcggagaatggctcg
gcggcgggcggcgccaacaatggcggcgctgctcagcagcatgcggcg
ccggcgatccgggagcaggaccggctgatgccgatcgcgaacgtgatc
cgcatcatgcgccgtgcgctccctgcccacgccaagatctccgacgac
gccaaggaggcgattcaggaatgcgtgtccgagttcatcagcttcgtc
accggcgaggccaacgaacggtgccgcatgcagcaccgcaagaccgtc
aacgccgaagacatcgtgtgggccctaaaccgcctcggcttcgacgac
tacgtcgtgcccctcagcgtcttcctgcaccgcatgcgcgagttcgag
ggcgacgcgcgcggcgtcgggctcgtcccgggggccgccccatcgcgc
ggcggcgaccaccacccgcactccatgtcgccagcggcgatgctcaag
tcccgcgggccagtctccggagccgccatgctaccgcaccaccaccac
caccacgacatgcagatgcacgccgccatgtacgggggaacggccgtg
cccccgccggccgggcctcctcaccacggcgggttcctcatgccacac
ccacagggtagtagccactacctgccttacgcgtacgagcccacgtac
ggcggtgagcacgccatggctgcatactatggaggcgccgcgtacgcg
cccggcaacggcgggagcggcgacggcagtggcagtggcggcggtggc
gggagcgcgtcgcacacaccgcagggcagcggcggcttggagcacccg
cacccgttcgcgtacaagtag

### Maize Chimeric LEC1 (Maize A-Wheat B (Underlined)-Maize C)

(SEQ ID NO: 13)
MDSSSFLPAAGAENGSAAGGANNGGAAQQHAAPAIREQDRLMPIANVI
RIMRRALPAHAKISDDAKEAIQECVSEFISFVTGEANERCRMQHRKTV
NAEDIVWALNRLGFDDYVVPLSVFLHRMREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

### Signature Sequence of B Domain

| | |
|---|---|
| MPIANVI | (SEQ ID NO: 14) |

Arabidopsis LEC1 Amino Acid Sequence with B-Domain Underlined (Signature Motif Bolded)

(SEQ ID NO: 15)
MTSSVIVAGAGDKNNGIVVQQQPPCVAREQDQYMPIANVIRIMRKTLP
SHAKISDDAKETIQECVSEYISFVTGEANERCQREQRKTITAEDILWA
MSKLGFDNYVDPLTVFINRYREIETDRGSALRGEPPSLRQTYGGNGIG
FHGPSHGLPPPGPYGYGMLDQSMVMGGGRYYQNGSSGQDESSVGGGSS
SSINGMPAFDHYGQYK

Maize LEC1 Nucleotide Sequence with B-Domain Underlined

(SEQ ID NO: 16)
atggactccagcagcttcctccctgccgccggcgcggagaatggctcg
gcggcgggcggcgccaacaatggcggcgctgctcagcagcatgcggcg
ccggcgatccgcgagcaggaccggctgatgccgatcgcgaacgtgatc
cgcatcatgcggcgcgtgctgccggcgcacgccaagatctcggacgac
gccaaggagacgatccaggagtgcgtgtcggagtacatcagcttcatc
acgggggaggccaacgagcggtgccagcgggagcagcgcaagaccatc
accgccgaggacgtgctgtgggccatgagccgcctcggcttcgacgac
tacgtcgagccgctcggcgcctacctccaccgctaccgcgagttcgag
ggcgacgcgcgcggcgtcgggctcgtcccgggggccgccccatcgcgc
ggcggcgaccaccacccgcactccatgtcgccagcggcgatgctcaag
tcccgcgggccagtctccggagccgccatgctaccgcaccaccaccac
caccacgacatgcagatgcacgccgccatgtacgggggaacggccgtg
cccccgccggccgggcctcctcaccacggcgggttcctcatgccacac
ccacagggtagtagccactacctgccttacgcgtacgagcccacgtac
ggcggtgagcacgccatggctgcatactatggaggcgccgcgtacgcg
cccggcaacggcgggagcggcgacggcagtggcagtggcggcggtggc
gggagcgcgtcgcacacaccgcagggcagcggcggcttggagcacccg
cacccgttcgcgtacaagtag

Maize LEC1 Amino Acid Sequence with B-Domain Underlined

(SEQ ID NO: 17)
MDSSSFLPAAGAENGSAAGGANNGGAAQQHAAPAIREQDRLMPIANVI
RIMRRVLPAHAKISDDAKETIQECVSEYISFITGEANERCQREQRKTI
TAEDVLWAMSRLGFDDYVEPLGAYLHRYREFEGDARGVGLVPGAAPSR
GGDHHPHSMSPAAMLKSRGPVSGAAMLPHHHHHHDMQMHAAMYGGTAV
PPPAGPPHHGGFLMPHPQGSSHYLPYAYEPTYGGEHAMAAYYGGAAYA
PGNGGSGDGSGSGGGGGSASHTPQGSGGLEHPHPFAYK

### Wheat LEC1 B Domain

(SEQ ID NO: 18)
cgggagcaggaccggctgatgccgatcgcgaacgtgatccgcatcatg
cgccgtgcgctccctgcccacgccaagatctccgacgacgccaaggag
gcgattcaggaatgcgtgtccgagttcatcagcttcgtcaccggcgag
gccaacgaacggtgccgcatgcagcaccgcaagaccgtcaacgccgaa
gacatcgtgtgggccctaaaccgcctcggcttcgacgactacgtc
gtgcccctcagcgtcttcctgcaccgcatgcgc

### Wheat LEC1 Amino Acid B Domain

(SEQ ID NO: 19)
REQDRLMPIANVIRIMRRALPAHAKISDDAKEAIQECVSEFISFVTGE
ANERCRMQHRKTVNAEDIVWALNRLGFDDYVVPLSVFLHRMR_{—}

Wheat LEC1 Nucleotide Sequence, with B Domain Underlined

(SEQ ID NO: 20)
atggagaacgacggcgtccccaacggaccagcggcgccggcacctacc
caggggacgccggtggtgcgggagcaggaccggctgatgccgatcgcg
aacgtgatccgcatcatgcgccgtgcgctccctgcccacgccaagatc
tccgacgacgccaaggaggcgattcaggaatgcgtgtccgagttcatc
agcttcgtcaccggcgaggccaacgaacggtgccgcatgcagcaccgc
aagaccgtcaacgccgaagacatcgtgtgggccctaaaccgcctcggc
ttcgacgactacgtcgtgcccctcagcgtcttcctgcaccgcatgcgc
gaccccgaggcggggacaggtggtgccgctgcaggcgacagccgcgcc
gtgacgagtgcgcctccccgcgcggccccgcccgtgatccacgccgtg
ccgctgcaggctcagcgcccgatgtacgcgcccccggctccgttgcag
gttgagaatcagatgcagcggcctgtgtacgctcccccggctccggtg
caggttcagatgcagcggggcatctatgggccccgggctccagtgcac
gggtacgccgtcggaatggcgcccgtgcgggccaacgtcggcgggcag
taccaggtgttcggcggagagggtgtcatggcccagcaatactacggg
tacgggtacgaggaaggagcgtacggcgcaggtagcagcaacggagga
gccgccattggcgacgaggagagctcgtccaacggcgtgccggcaccg
ggggagggcatgggggagccagagccagagccagcagcagaagaatcg
catgacaagcccgtccaatctggctag

Wheat LEC1 Amino Acid Sequence with B-Domain Underlined

(SEQ ID NO: 21)
MENDGVPNGPAAPAPTQGTPVVREQDRLMPIANVIRIMRRALPAHAKI
SDDAKEAIQECVSEFISFVTGEANERCRMQHRKTVNAEDIVWALNRLG
FDDYVVPLSVFLHRMRDPEAGTGGAAAGDSRAVTSAPPRAAPPVIHAV
PLQAQRPMYAPPAPLQVENQMQRPVYAPPAPVQVQMQRGIYGPRAPVH
GYAVGMAPVRANVGGQYQVFGGEGVMAQQYYGYGYEEGAYGAGSSNGG
AAIGDEESSSNGVPAPGEGMGEPEPEPAAEESHDKPVQSG

### B-Domain for Maize LEC1 Variant 9

(SEQ ID NO: 22)
cgcgagcaggaccggctgatgccgatcgcgaacgtggcccgcatcatg
aagcgcgtggtgccggcgcacgcccgcgtgteggacgacgccaaggag
acgctgcaggagtgcgtgtcggagtacatcagettcatcacgggggag
gccaacgagcggtgccgccaggagcagcgcaagaccctgaccgccgag
gacatcctgttcgccatgagccgcctcggcttcgacgactacgtcgag
ccgctcggcgcctacctccaccgctaccgc

### Maize LEC1 Variant 9 B-Domain

(SEQ ID NO: 23)
REQDRLMPIA NVARIMKRVV PAHARVSDDA KETLQECVSE
YISFITGEAN ERCRQEQRKT LTAEDILFAM SRLGFDDYVE
PLGAYLHRYR

### B-Domain for Maize LEC1 Variant 12

(SEQ ID NO: 24)
cgcgagcaggaccggctgatgccgatcgcgaacgtggcccgcctgatg
aagcgcgtgatcccggcgcacgcccgcgtggccgacgacgccaaggag
acgctgcaggagtgcgtgtcggagttcatcagcttcatcacgagcgag
gccagcgagcggtgccgccaggagcagcgcaagaccatcaccgccgag
gacctcctgtgggccctgagccgcctcggcttcgacgactacgtcgag
ccgctcggcgcctacctccaccgctaccgc

### Maize LEC1 Variant 12 B-Domain

(SEQ ID NO: 25)
REQDRLMPIA NVARLMKRVI PAHARVADDA KETLQECVSE
FISFITSEAS ERCRQEQRKT ITAEDLLWAL SRLGFDDYVE
PLGAYLHRYR

### B-Domain for Maize LEC1 Variant 15

(SEQ ID NO: 26)
cgcgagcaggaccggctgatgccgatcgcgaacgtggcccgcatcatg
cggcgcgtgctgccggcgcacgccaagatctcggacgacgccaaggag
acgatccaggagtgcgtgtcggagtacatcagcttcatcacgggggag
gccaacgagcggtgccagcgggagcagcgcaagaccatcaccgccgag
gacgtgctgtgggccatgagccgcctcggcttcgacgactacgtcgag
ccgctcggcgcctacctccaccgctaccgc_

### Maize LEC1 Variant 15 B-Domain

(SEQ ID NO: 27)
REQDRLMPIA NVARIMRRVL PAHAKISDDA KETIQECVSE
YISFITGEAN ERCQREQRKT ITAEDVLWAM SRLGFDDYVE
PLGAYLHRYR

### B-Domain for Maize LEC1 Variant 17

(SEQ ID NO: 28)
Cgcgagcaggaccggctgatgccgatcgcgaacatcatccgcatcatg
cggcgcgtgctgccggcgcacgccaagatctcggacgacgccaaggag
acgatccaggagtgcgtgtcggagtacatcagcttcatcacgggggag
gccaacgagcggtgccagcgggagcagcgcaagaccatcaccgccgag
gacgtgctgtgggccatgagccgcctcggcttcgacgactacgtcgag
ccgctcggcgcctacctccaccgctaccgc

### Maize LEC1 Variant 17 B-Domain

(SEQ ID NO: 29)
REQDRLMPIA NIIRIMRRVL PAHAKISDDA KETIQECVSE
YISFITGEAN ERCQREQRKT ITAEDVLWAM SRLGFDDYVE
PLGAYLHRYR

This invention can be better understood by reference to the following non-limiting examples.

### EXAMPLES

The present invention is further defined in the following Examples, in which parts and percentages are by weight and degrees are Celsius, unless otherwise stated. The disclosure of each reference set forth herein is incorporated herein by reference in its entirety.

### Example 1

### Vector Construction

Standard restriction fragment preparation and ligation techniques were used to position each LEC1 gene between the LTP2 promoter (U.S. Pat. No. 5,525,716) and a potato PIN II terminator. Each completed gene cassette was flanked by Gateway™ (Invitrogen) homologous recombination sites ATT L1 and ATT L2. These were used to mobilize the LEC1 gene expression cassettes into Gateway™-modified pSB11-derived T-DNA vectors (Japan Tobacco). These T-DNA vectors contained a selectable marker (a Ubi::moPAT::PinII expression cassette consisting of the maize ubiquitin-1 promoter including the 5′-untranslated region and first intron, a maize-optimized PAT gene, U.S. Pat. No. 6,096,947 and potato PINII terminator). In some vectors, a screenable marker, the DS-RED2 gene (Clontech), under the control of the aleurone-specific END2 promoter and potato PINII terminator, was also added. Each confirmed T-DNA vector was transformed via electroporation into Agrobacterium tumefaciens LBA4404 (pSB1) cells and the resulting cointegrate plasmid confirmed by extensive restriction digest analysis. Constructs were introduced into maize Hi-II line using Agrobacterium-mediated transformation method as described previously. T0 plants were crossed with non-transgenic inbred lines to produce T1 seeds.

### Example 2

### Expression of Maize LEC1 Variants

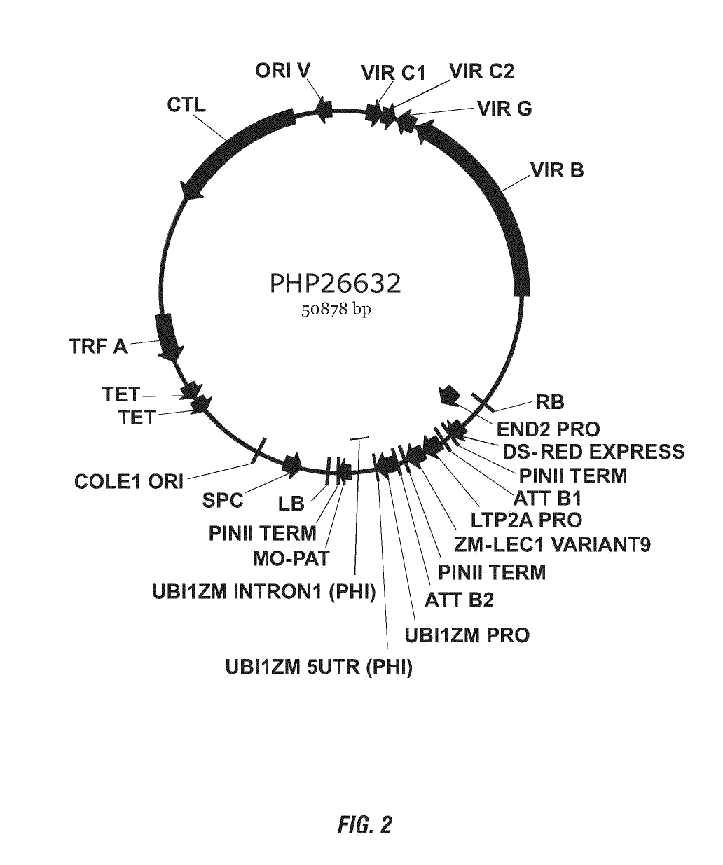

Maize LEC1 variant 9 (SEQ ID NO:4) was expressed under the LTP2 promoter and pinII terminator (FIG. 2) and introduced into maize via Agrobacterium-mediated transformation. A total of 12 transgenic events were generated and produced T1 seeds. For each event, transgenic kernel was separated from null kernel by red fluorescence marker, 10 transgenic kernels were compared to 10 null kernels from the same ear.

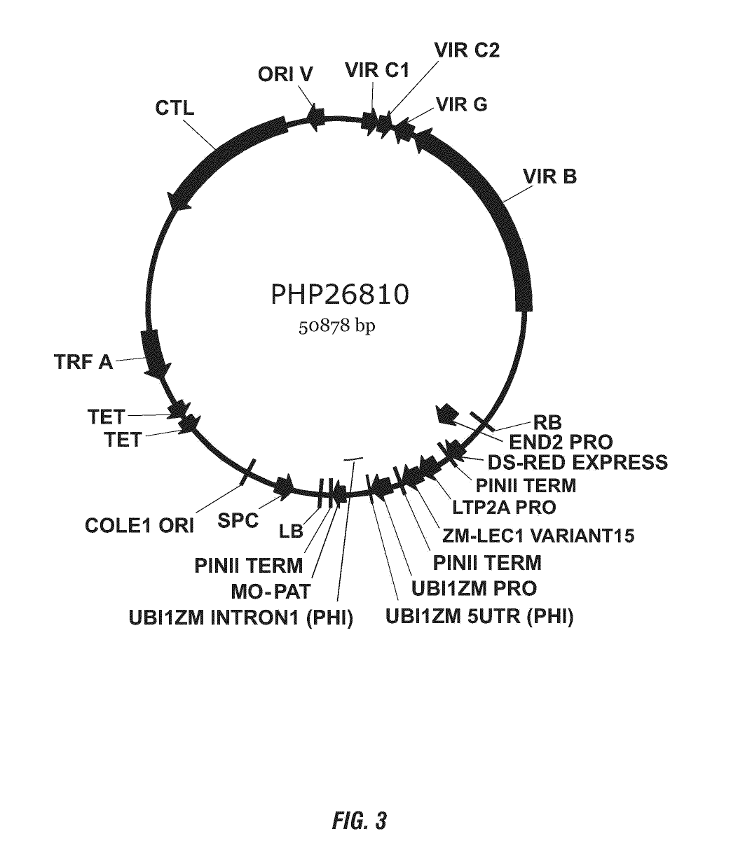

Similarly, maize LEC1 variant 15 (SEQ ID NO:8) was moved into an expression cassette containing a Ltp2 promoter and a PinII terminator. This cassette was linked to another cassette containing a red fluorescence protein expressed under an aleurone layer specific END2 promoter with a Pin II terminator. The red fluorescence protein was used as a visual marker to track transgenic LEC1 variant gene. The two expression cassettes were then subcloned adjacent to a Ubiquitin promoter:Mo-PAT expression cassette. The resulting expression cassettes flanked by T-DNA border sequences were then introduced into the Agrobacterium “super-binary” vector using electroporation, resulting in construct PHP26810 (FIG. 3). A total of 17 transgenic events were generated and produced T1 seeds. For each event, transgenic kernel was separated from null kernel by red fluorescence marker, 10 transgenic kernels were compared to 10 null kernels from the same ear.

### Example 3

### Expression of Maize-Wheat Chimeric LEC1

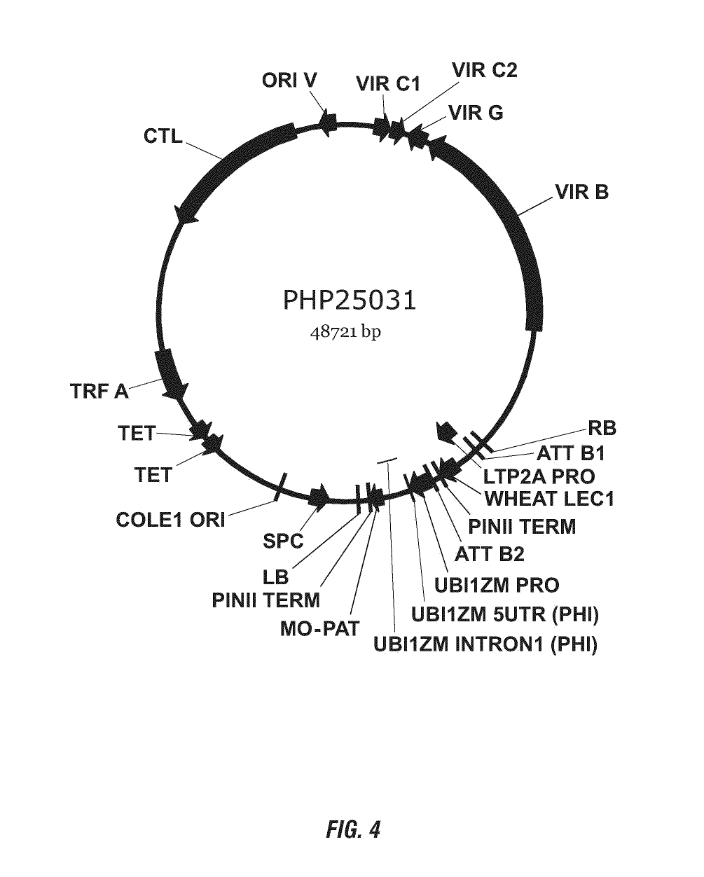

Wheat LEC1 was moved into an expression cassette containing a Ltp2 promoter and a PinII terminator. This cassette was then subcloned adjacent to a Ubiquitin promoter:Mo-PAT expression cassette. The resulting expression cassettes flanked by T-DNA border sequences were then introduced into the Agrobacterium “super-binary” vector using electroporation, resulting in construct PHP25031 (FIG. 4). Corn plant was transformed with super-binary vector containing two expression cassettes via Agrobacterium-mediated transformation. A total of 10 transgenic events were generated and produced T1 seeds. For each event, embryo was dissected from 10 kernels. Genotype of each kernel was determined by PCR using primers specific to Mo-PAT gene.

### Example 4

### Complementation Studies Showing the Ability of Maize LEC1 Variants to Complement an Arabidopsis lec1 Mutant

To test if maize LEC1 variants is functional as wild type LEC1, four maize LEC1 variants, variant 9 (SEQ ID NO:4), 12 (SEQ ID NO:6), 15 (SEQ ID NO:8), 17 (SEQ ID NO:10), and wild type maize LEC1 (SEQ ID NO:16 were cloned into binary vector linked to a constitutive SCP1 promoter (U.S. Pat. No. 6,072,050). The expression vectors were introduced into Agrobacteria through electroporation. Homozygous Arabidopsis lec1 mutant plants were rescued before silique drying and were transformed with 4 maize LEC1 variant constructs and wild type control using floral dip transformation method (Clough S J, Bent A F. Floral dip: a simplified method for Agrobacterium-mediated transformation of Arabidopsis thaliana. Plant J. 1998 December; 16(6):735-43). Homozygous lec1 mutant seeds do not germinate because their embryos are not tolerant to desiccation and died on drying. Expression of maize variants and wild type control in Arabidopsis lec1 mutant complements mutant and produced viable seeds, indicating that maize LEC1 variants function like the maize wild type LEC1. The data indicate that amino acid changes in MPIANVI motif do not affect LEC1 function.

### Example 5

### Expression of Maize LEC1 Variant 9 Increase Callus Growth and Transformation Efficiency

Maize LEC1 (SEQ ID NO:16) and LEC1 variant 9 (SEQ ID NO:4) were expressed under maize ubiquitin promoter. A ubiquitin driven GUS gene was also used as a control. These three constructs were each separately co-transformed into Hi-Type II embryos along with an expression cassette containing a Ubiquitin-driven maize codon optimized PAT gene fused to GFP. After selection on bialaphos the frequencies of bialaphos resistant GFP+ colonies were determined. As shown in Table 5, expression of maize LEC1 and LEC1 variant 9 increase transformation frequencies by 8-fold and 7.5-fold respectively compared to control. In addition to increasing transformation frequency, LEC1 greatly increased the growth rate of transformants.

TABLE 2
Effects of maize LEC1 and LEC1 variant
9 on transformation efficiency

| | % of embryos | Average | Transformation |
|---|---|---|---|
| Treatment | with Tx events | events/embryo | Frequency % |
| Ubi: GUS control | 6 | 1 | 6 |
| Ubi: LEC1 | 19 | 2.5 | 47.5 |
| Ubi: LEC1 variant 9 | 1 | 2.5 | 45 |

### Example 6

### Transformation and Regeneration of Maize Callus

Immature maize embryos from greenhouse or field grown High type II donor plants may be bombarded with a plasmid containing a LEC1 variant polynucleotide of the invention. The LEC1 variant polynucleotide may be operably linked to a constitutive promoter such as nos, or an inducible promoter, such as In2, plus a plasmid containing a selectable marker gene such as PAT (Wohlleben et al. (1988) Gene 70:25-37) that confers resistance to the herbicide Bialaphos fused to the Green Fluorescence protein. Transformation may be performed as follows.

The ears may be surface sterilized in 50% Chlorox bleach plus 0.5% Micro detergent for 20 minutes, and rinsed two times with sterile water. The immature embryos are then excised and placed embryo axis side down (scutellum side up), 25 embryos per plate. These embryos can be cultured on 560 L medium 4 days prior to bombardment in the dark. Medium 560 L is an N6-based medium containing Eriksson's vitamins, thiamine, sucrose, 2,4-D, and silver nitrate. On the same day as bombardment, the embryos are transferred to 560 Y medium for 4 hours and arranged within the 2.5-cm target zone. Medium 560Y is a high osmoticum medium (560L with high sucrose concentration).

A plasmid vector comprising a polynucleotide of the invention operably linked to the selected promoter will be constructed. This plasmid DNA plus plasmid DNA containing a PAT selectable marker will be precipitated onto 1.1 μm (average diameter) tungsten pellets using a CaCl₂ precipitation procedure as follows: 100 μl prepared tungsten particles (0.6 mg) in water, 20 μl (2 μg) DNA in TrisEDTA buffer (1 μg total), 100 μl 12.5 M CaCl₂, 40 μl 0.1 M spermidine.

Each reagent will be added sequentially to the tungsten particle suspension. The final mixture may be sonicated briefly. After the precipitation period, the tubes will be centrifuged briefly, liquid removed, washed with 500 ml 100% ethanol, and centrifuged again for 30 seconds. Again the liquid will be removed, and 60 μl 100% ethanol will be added to the final tungsten particle pellet. For particle gun bombardment, the tungsten/DNA particles will be briefly sonicated and 5 μl spotted onto the center of each macrocarrier and will be allowed to dry about 2 minutes before bombardment.

The sample plates may be bombarded at a distance of 8 cm from the stopping screen to the tissue, using a Dupont biolistics helium particle gun. All samples should receive a single shot at 650 PSI, with a total of ten aliquots taken from each tube of prepared particles/DNA.

Four to 12 hours post bombardment, the embryos will be moved to 560P (a low osmoticum callus initiation medium similar to 560L but with lower silver nitrate), for 3-7 days, then transferred to 560R selection medium, an N6 based medium similar to 560P containing 3 mg/liter Bialaphos, and subcultured every 2 weeks. Multicellular GFP cell clusters may become visible after two weeks and their numbers will be periodically recorded. After approximately 10 weeks of selection, selection-resistant GFP positive callus clones will be sampled for PCR and activity of the polynucleotide of interest. Positive lines will be transferred to 288J medium, an MS-based medium with lower sucrose and hormone levels, to initiate plant regeneration. Following somatic embryo maturation (2-4 weeks), well-developed somatic embryos will be transferred to medium for germination and transferred to the lighted culture room. Approximately 7-10 days later, developing plantlets will be transferred to medium in tubes for 7-10 days until plantlets are well established. Plants will then be transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Plants will be monitored for expression of the polynucleotide of interest.

### Example 7

### Ectopic Expression of LEC1 Variant to Induce Somatic Embryogenesis

Using the genotype High type II as an example, embryos can be isolated and cultured on 560L medium for 3-5 days. Four to twelve hours before bombardment these embryos will be transferred to high osmotic 560Y medium. Expression cassettes containing the LEC1 variant polynucleotide cDNA can then be co-introduced into the scutella of these embryos along with an expression cassette containing the Pat gene fused to the Green Fluorescent protein using methods described in herein. Embryos from a single ear can be divided evenly between treatments. Four to 12 hours following bombardment embryos can then be transferred back to a low osmoticum callus initiation medium (560P) and incubated in the dark at 26° C. After 3-7 days of culture these embryos will be moved to 560R selection medium. Cultures will then be transferred every two weeks until transformed colonies appear. Cultures will also be examined microscopically for GFP expression and for the ability of the LEC1 variant to stimulate adventive embryo formation. This will become apparent when the cultures are compared to controls (transformed without the LEC1 variant polynucleotide cDNA or non-induced).

### Example 8

### LEC1 Variant Introduced Via Bombardment and its Effect on Transformation Frequency

A series of expression cassettes can be made to evaluate the effects of LEC1 variant expression on maize transformation. The maize LEC1 variant polynucleotide will be placed under the control of the In2 promoter (weakly induced with the auxin levels used under normal culture conditions and strongly-induced with safener), the barley NUC1 promoter (expressed strongly in the nucellus), the Ubiquitin promoter (strongly expressed constitutively), and the nos promoter (weakly expressed constitutively). A frame-shift version of the In2:LEC1 cassette can be made along with an In2:ZM-NF-YB (designated as In2:HAP3 henceforth) construct (The maize ZM NF-YB is non-LEC1 type of HAP3 transcriptional activator (Li et al Nucleic Acids Res. 20:1087-1091) for use as negative controls). All of these constructs will be co-bombarded with the Pat˜GFP fusion construct (designated as PAT˜GFP) into high type II embryos as described in Example 6. Also, as in Example 6, immature embryos can be harvested from separate ears, and the embryos from each ear will be divided equally between treatments to account for ear-to-ear variability (for example, in an experiment comparing a control plasmid with this same plasmid+LEC1 variant, one-half the total embryos from each ear would be used for each treatment). In some cases the control treatment may contain the Pat˜GFP construct co-bombarded with GUS. Transformation frequencies will be determined by counting the numbers of embryos with large multicellular GFP-positive cells clusters using a GFP microscope, and representing these as a percentage of the original number of embryos bombarded. No distinction will be made between embryos with single or multiple events. LEC1 variant expression cassettes may be evaluated for their ability to increase transformation frequencies over the control treatment.

Increasing the promoter strength driving a LEC1 variant expression may increase transformation frequencies. Within these treatments there may also be an increase in the overall frequency of large, rapidly growing calli. For the control treatments, the frequencies of large, vigorous GFP+ calli relative to the starting number of embryos may be low. The overall frequencies of large, vigorous calli may be evaluated.

### Example 9

### LEC1 Variant Introduced Via Agrobacterium and its Effect on Transformation Frequency

Agrobacterium strains containing the superbinary plasmids described in Example 7 can be used to transform High type II embryos. Briefly, colonies containing the engineered Agrobacterium can be grown to log phase in minimal A medium. Log phase cells may be collected by centrifugation and resuspended in 561Q medium (N6 salts, Eriksson's vitamins, 1.5 mg/l 2,4-D, 68.5 g/l sucrose, 36 g/l glucose, plus 20 mg/l acetosyringone). Immature embryos, 1.5-2 mm in length, may be excised and immersed in this solution at a concentration of 5×10⁸ bacterial cells/ml. Embryos may be vortexed in this medium and allowed to sit for 5 minutes. The embryos would then be removed and placed on 562P medium (560P medium with 100 mM acetosyringone) and incubated at 20° C. for 3 days. Embryos would then be moved again to 563N medium (an agar solidified medium similar to 560P with 100 mg/l carbenicillin, 0.5 g/l MES and reduced 2,4-D) and cultured at 28° C. for 3 days. Embryos may then be moved to 563O medium (563N medium with 3 mg/l bialaphos) and transferred thereafter every 14 days to fresh 563O medium.

Bialaphos resistant GFP+ colonies are counted using a GFP microscope and transformation frequencies will be determined as described in Example 8. Similar to particle gun experiments, transformation frequencies should be greatly increased in the LEC1 variant treatment.

### Example 10

### Recovering Transformants Using LEC1 Variants Expression Under Reduced Auxin Levels or in the Absence of Auxins in the Medium, and in the Absence of Herbicide or Antibiotic Selection

To determine if LEC1 variants may be used in a positive selection scheme. Scheme particle gun transformation experiments will be initiated as described in Example 6 and transformants will be selected on medium with normal auxin levels, or on medium with reduced or no auxin, or visually (using GFP) on medium without bialaphos. Transformation frequencies will be based on the numbers of embryos with one or more multicellular GFP positive cell clusters. In the first experiment to test this concept, there will be two treatment variables. The first is immature embryos bombarded with the control plasmid (UBI:PAT˜GFP) or with UBI:PAT˜GFP+In2:LEC1. The second variable will be the bombarded embryos divided onto either normal bialaphos-containing selection medium (with normal auxin levels of 2 mg/l 2,4-D), or medium with no bialaphos and reduced 2,4-D levels (0.5 mg/l). It is predicted that, on bialaphos selection the LEC1 variant treatment will result in a higher transformation frequency than the control. It is also likely that the low auxin medium (0.5 mg/l 2,4-D) will result in reduced growth rates. LEC1 variant expression will likely compensate for the low auxin environment, thus providing a growth advantage to the transgenic colonies, and maintaining the efficiency of transformant recovery in the same range as the LEC1 variant/bialaphos-selected treatment.

On medium completely devoid of auxin, colonies will likely only be observed in the LEC1 variant treatment. In this experiment, immature embryos will be bombarded with either the control plasmid (UBI:PAT˜GFP) or with UBI:PAT˜GFP+In2:LEC1, and then plated either onto 3.0 mg/l bialaphos, 2.0 mg/l 2,4-D medium or onto no-bialaphos, no 2,4-D medium (in this latter treatment, wild-type maize callus will not exhibit embryogenic growth). It is expected that the LEC1 variant polynucleotide will show increased transformation over the control plasmid on normal auxin-containing, bialaphos selection medium. Also, it is likely that no transformants will be recovered with the control plasmid on medium devoid of exogenous auxin. It is also likely that in the LEC1 variant treated embryos, transformants will be recovered at a frequency higher than the control plasmid on bialaphos selection).

Even on auxin-containing medium, the LEC1 variant polynucleotide in combination with GFP+ expression can be used to recover transformants without chemical selection. For example, under these conditions the recovery of transformants will be relatively efficient but this will require more diligence than the low- or no-auxin treatments above to separate the GFP-expressing colonies from the growing callus population.

### Example 11

### LEC1 Variant May Improve the Embryogenic Phenotype and Regeneration Capacity of Inbreds

Immature embryos from the inbred PHP38 can be isolated, cultured and transformed, as described in Example 6, with the following changes. Embryos would be initially cultured on 601H medium (a MS based medium with 0.1 mg/l zeatin, 2 mg/l 2,4-D, MS and SH vitamins, proline, silver nitrate, extra potassium nitrate, casein hydrolysate, gelrite, 10 g/l glucose and 20 g/l sucrose). Prior to bombardment embryos will be moved to a high osmoticum medium (modified Duncan's with 2 mg/l 2,4-D and 12% sucrose). Post bombardment, embryos will be moved to 601H medium with 3 mg/l bialaphos for two weeks. Embryos will then be moved to 601H medium without proline and casein hydrolysate with 3 mg/l bialaphos and will be transferred every two weeks. Transformation frequency may be determined by counting the numbers of bialaphos resistant GFP-positive colonies. Colonies may also be scored on whether they had an embryogenic (regenerable) or non-embryogenic phenotype. The LEC1 variant polynucleotide will likely increase transformation frequency and improve the regenerative potential of the callus. For example, a balanced experiment (the embryos from each harvested ear were divided equally between treatments) may be conducted in which PHP38 immature embryos would be bombarded with the control plasmid (UBI::PAT˜GFP::pinII) in one treatment, with the UBI::PAT˜GFP::pinII plasmid+In2::LEC1, or with the UBI::PAT˜GFP::pinII plasmid+nuc1::LEC1 variant (a maize nucellus-specific promoter driving LEC1 variant expression). The frequency of GFP+ calli growing on bialaphos-containing media (relative to the starting number of embryos) may be determined 6 weeks after bombardment.

### Example 12

### Transient Expression of the LEC1 Variant polynucleotide Product to Induce Somatic Embryogenesis

It may be desirable to “kick start” somatic embryogenesis by transiently expressing the LEC1 variant polynucleotide product. This can be done by delivering LEC1 variant 5′ capped polyadenylated RNA, expression cassettes containing LEC-1 DNA, or LEC1 protein. All of these molecules can be delivered using a biolistics particle gun. For example 5′ capped polyadenylated LEC1 variant RNA can easily be made in vitro using Ambion's mMessage mMachine kit. Following the procedure outline above RNA is co-delivered along with DNA containing an agronomically useful expression cassette. The cells receiving the RNA will immediately form somatic embryos and a large portion of these will have integrated the agronomic gene. Plants regenerated from these embryos can then be screened for the presence of the agronomic gene.

### Example 13

### Use of the LEC1 Variant to Induce Apomixis

Maize expression cassettes directing LEC1 variant expression to the inner integument or nucellus can easily be constructed. An expression cassette directing expression of the LEC1 variant polynucleotide to the nucellus can be made using the barley Nuc1 promoter. Embryos would be co-bombarded with the selectable marker PAT fused to the GFP gene along with the nucellus specific LEC1 variant expression cassette described above. Both inbred (PHP38) and GS3 transformants may then be obtained and regenerated using techniques well known in the art. Transformation frequencies may also be increased over the control using the nuc1:LEC1 polynucleotide.

It is anticipated that the regenerated plants will then be capable of producing de novo embryos from LEC1 variant expressing nucellar cells. This is complemented by pollinating the ears to promote normal central cell fertilization and endosperm development. In another variation of this scheme, nuc1:LEC1 variant transformations could be done using a FIE-null genetic background which would promote both de novo embryo development and endosperm development without fertilization (see Ohad et al. 1999 The Plant Cell 11:407-415; also pending U.S. application Ser. No. 60/151,575 filed Aug. 31, 1999). Upon microscopic examination of the developing embryos it may be determined that apomixis has occurred by the presence of embryos budding off the nucellus. In yet another variation of this scheme the LEC1 variant polynucleotide could be delivered as described above into a homozygous zygotic-embryo-lethal genotype. Only the adventive embryos produced from somatic nucellus tissue would develop in the seed.

### Example 14

### LEC1 Variant Expression May Increase Growth Rates, which could be Used as a Screening Criterion for Positive Selection of Transformants

An experiment can be performed to compare the In2 and nos promoters. Based on our experience with these two promoters driving other genes, the In2 promoter (in the absence of an inducer other than auxin from the medium) will drive expression at very low levels. The nos promoter has been shown to drive moderately-low levels of transgene expression (approximately 10- to 30-fold lower than the maize ubiquitin promoter, but still stronger than In2 under the culture conditions used in this experiment). One control treatment can be used in this experiment, the UBI:PAT˜GFPmo:pinII construct by itself (with no LEC1). Hi-II immature embryos will be bombarded as previously described, and transgenic, growing events may be scored at 3 and 6 weeks for transformation frequencies.

Within these treatments there may also be an increase in the overall frequency of large, rapidly growing calli, relative to the control treatment. For this data, the fresh weight of transformed calli will be recorded 2 months after bombardment. Assuming that all the transgenic events started as single transformed cells within a few days after bombardment, these weights would represent the relative growth rate of these transformants during this period (all tissue would be sub-cultured and weighed for each transformant; mean weights and standard deviations would be calculated for each treatment). For the control treatment, the mean transformant weight after two months should be lower than that of the In2:LEC1 variant and nos:LEC1 variant treatments. If the control treatment is set at a relative growth value of 1.0, this means that transformants in the In2:LEC1 variant and nos:LEC1 variant treatments will grow several-fold faster than the control.

### Example 15

### The Use of LEC1 Variants Polynucleotide as a Positive Selection System for Wheat Transformation and for Improving the Regeneration Capacity of Wheat Tissues

### Plant Material

Seeds of wheat Hybrinova lines NH535 and BO 014 will be sown into soil in plug trays for vernalisation at 6° C. for eight weeks. The vernalized seedlings can be transferred in 8″ pots and grown in a controlled environment room. The growth conditions would be; 1) soil composition: 75% L&P fine-grade peat, 12% screened sterilized loam, 10% 6 mm screened, lime-free grit, 3% medium grade vermiculite, 3.5 kg Osmocote per m³ soil (slow-release fertiliser, 15-11-13 NPK plus micronutrients), 0.5 kg PG mix per m³ (14-16-18 NPK granular fertiliser plus micronutrients, 2) 16 h photoperiod (400 W sodium lamps providing irradiance of ca. 750 μE s⁻¹ m⁻²), 18 to 20° C. day and 14 to 16° C. night temperature, 50 to 70% relative air humidity and 3) pest control: sulphur spray every 4 to 6 weeks and biological control of thrips using Amblyseius caliginosus (Novartis BCM Ltd, UK).

### Isolation of Explants and Culture Initiation

Two sources of primary explants will be used; scutellar and inflorescence tissues. For scutella, early-medium milk stage grains containing immature translucent embryos will be harvested and surface-sterilized in 70% ethanol for 5 min and 0.5% hypochlorite solution for 15-30 min. For inflorescences, tillers containing 0.5-1.0 cm inflorescences will be harvested by cutting below the inflorescence-bearing node (the second node of a tiller). The tillers will be trimmed to approximately 8-10 cm length and surface-sterilized as above with the upper end sealed with Nescofilm (Bando Chemical Ind. Ltd, Japan).

Under aseptic conditions, embryos of approximately 0.5-1.0 mm length will be isolated and the embryo axis removed. Inflorescences would then be dissected from the tillers and cut into approximately 1 mm pieces. Thirty scutella or 1 mm inflorescence explants would then be placed in the center (18 mm target circle) of a 90 mm Petri dish containing MD0.5 or L7D2 culture medium. Embryos should be placed with the embryo-axis side in contact with the medium exposing the scutellum to bombardment whereas inflorescence pieces are placed randomly. Cultures would then be incubated at 25±° C. in darkness for approximately 24 h before bombardment. After bombardment, explants from each bombarded plate would then be spread across three plates for callus induction.

### Culture Media

The standard callus induction medium for scutellar tissues (MD0.5) should consist of solidified (0.5% Agargel, Sigma A3301) modified MS medium supplemented with 9% sucrose, 10 mg l⁻¹ AgNO₃ and 0.5 mg l⁻¹ 2,4-D (Rasco-Gaunt et al., 1999). Inflorescence tissues will be cultured on L7D2 which consisted of solidified (0.5% Agargel) L3 medium supplemented with 9% maltose and 2 mg l⁻¹ 2,4-D (Rasco-Gaunt and Barcelo, 1999). The basal shoot induction medium, RZ contains L salts, vitamins and inositol, 3% w/v maltose, 0.1 mg l⁻¹ 2,4-D and 5 mg l⁻¹ zeatin (Rasco-Gaunt and Barcelo, 1999). Regenerated plantlets will be maintained in RO medium with the same composition as RZ, but without 2,4-D and zeatin.

### DNA Precipitation Procedure and Particle Bombardment

Submicron gold particles (0.6 μm Micron Gold, Bio-Rad) will be coated with a plasmid containing the maize In-2:LEC1 construct following the protocol modified from the original Bio-Rad procedure (Barcelo and Lazzeri, 1995). The standard precipitation mixture consists of 1 mg of gold particles in 50 μl SDW, 50 μl of 2.5 M calcium chloride, 20 μl of 100 mM spermidine free base and 5 μl DNA (concentration 1 μg μl⁻¹). After combining the components, the mixture will be vortexed and the supernatant discarded. The particles would then be washed with 150 μl absolute ethanol and finally resuspended in 85 μl absolute ethanol. The DNA/gold ethanol solution may be kept on ice to minimize ethanol evaporation. For each bombardment, 5 μl of DNA/gold ethanol solution (ca. 60 μg gold) may be loaded onto the macrocarrier.

Particle bombardments may be carried out using DuPont PDS 1000/He gun with a target distance of 5.5 cm from the stopping plate at 650 psi acceleration pressure and 28 in. Hg chamber vacuum pressure.

### Regeneration of Transformants

For callus induction, bombarded explants will be distributed over the surface of the medium in the original dish and two other dishes and cultured at 25±1° C. in darkness for three weeks. Development of somatic embryos from each callus may be periodically recorded. For shoot induction, calluses would be transferred to RZ medium and cultured under 12 h light (250 μE s⁻¹ m⁻², from cool white fluorescent tubes) at 25±1° C. for three weeks for two rounds. All plants regenerating from the same callus will be noted. Plants growing more vigorously than the control cultures will be potted in soil after 6-9 weeks in R0 medium. The plantlets will be acclimatized in a propagator for 1-2 weeks. Thereafter, the plants will be grown to maturity under growth conditions described above.

DNA Isolation from Callus and Leaf Tissues

Genomic DNA may be extracted from calluses or leaves using a modification of the CTAB (cetyltriethylammonium bromide, Sigma H5882) method described by Stacey and Isaac (1994). Under that method, approximately 100-200 mg of frozen tissues is ground into powder in liquid nitrogen and homogenised in 1 ml of CTAB extraction buffer (2% CTAB, 0.02 M EDTA, 0.1 M Tris-Cl pH 8, 1.4 M NaCl, 25 mM DTT) for 30 min at 65° C. The homogenized samples are allowed to cool at room temperature for 15 min before a single protein extraction with approximately 1 ml 24:1 v/v chloroform:octanol is done. These samples are centrifuged for 7 min at 13,000 rpm and the upper layer of supernatant collected using wide-mouthed pipette tips. DNA is precipitated from the supernatant by incubation in 95% ethanol on ice for 1 h. The DNA threads are spooled onto a glass hook, washed in 75% ethanol containing 0.2 M sodium acetate for 10 min, air-dried for 5 min and resuspended in TE buffer. Five μl RNAse A is added to the samples and incubated at 37° C. for 1 h.

For quantification of genomic DNA, gel electrophoresis may be performed using an 0.8% agarose gel in 1×TBE buffer. One microlitre of the samples can be fractionated alongside 200, 400, 600 and 800 ng μl⁻¹λ uncut DNA markers.

### Polymerase Chain Reaction (PCR) Analysis

The presence of the maize LEC1 polynucleotide may be analyzed by PCR using 100-200 ng template DNA in a 30 ml PCR reaction mixture containing 1× concentration enzyme buffer (10 mM Tris-HCl pH 8.8, 1.5 mM magnesium chloride, 50 mM potassium chloride, 0.1% Triton X-100), 200 μM dNTPs, 0.3 μM primers and 0.022 U TaqDNA polymerase (Boehringer Mannheim). Thermocycling conditions would be as follows (30 cycles): denaturation at 95° C. for 30 s, annealing at 55° C. for 1 min and extension at 72° C. for 1 min. One skilled in the art can design appropriate use primer sequences.

### Results

Following experiments to show increased regeneration capacity and improvement of maize transformation frequencies by expression of LEC1 variants, the polynucleotide will then be introduced into wheat scutellar and inflorescence explants, driven by the maize In2 promoter. Both tissues are useful for wheat transformation.

Subsequent to the induction of somatic embryos from both tissues after three weeks on a 2,4-D-containing induction medium, calluses will be assessed prior to transfer onto shoot regeneration medium. Callus assessment involves: a) scoring calluses as 0=non-embryogenic callus, 1=25%, 2=25-50%, 3=50-75%, 4=75-100% of callus surface embryogenic, and b) determining embryogenic capacity expressed in percentage as the number of embryogenic calluses/total number of calluses (scutella or inflorescence) assessed.

### Scutellum Calluses

After callus induction and assessment, calluses may be transferred onto shoot induction media for a total of six weeks. Shoot regeneration of calluses is determined, as the number of shoot regenerating calluses/total number of calluses assessed (expressed as percentages). Shoot regeneration of cultures corresponds with the quality and quantity of somatic embryos produced in each callus. Hence, regeneration of LEC1 variant-bombarded and control callus tissues of line NH535 will likely not be significantly different. However, regeneration of LEC1 variant-bombarded calluses of wheat should be significantly improved in comparison with the control

To test the suitability of a LEC1 variant as a positive selection system for wheat, sample tissues from vigorous calluses may be analyzed for the presence of LEC1 variant sequences. An appropriate sample would be forty-one calluses. The results would likely show that calluses were PCR positive. It is likely that transformed lines can be identified without selection at frequencies comparable with conventional selection systems such as herbicide- and antibiotic-resistance systems (e.g. bar, nptII) applied in wheat transformation where selection ‘escape’ frequencies are commonly high and variable.

### Inflorescence Calluses

The use of inflorescence tissues as explants for the tissue culture and transformation of wheat offer several advantages over seed explants such as scutella (Rasco-Gaunt and Barcelo, 1999). However, responses of these tissues to culture are highly genotype-dependent and calluses are often non-regenerative despite having a ‘highly-embryogenic’ appearance. Hence, LEC1 variants can be introduced into inflorescence tissues to see whether regeneration could be enhanced on a poorly regenerating line such BO 014.

Shoot regeneration may be significantly improved in LEC1 variant-bombarded tissues, although callus quality may appear similar in bombarded and control tissues.

### Example 16

### Expression of Chimeric Genes in Dicot Cells

The LEC1 variant polynucleotide may also be used to improve the transformation of soybean. An experiment demonstrating this result would involve the introduction of the construct consisting of the In2 promoter and LEC1 variant coding region into embryogenic suspension cultures of soybean by particle bombardment using essentially the methods described in Parrott, W. A., L. M. Hoffman, D. F. Hildebrand, E. G. Williams, and G. B. Collins, (1989) Recovery of primary transformants of soybean, Plant Cell Rep. 7:615-617. This method with appropriate modifications is described below.

Remove seed from pods when the cotyledons are between 3 and 5 mm in length. The seeds are then sterilized in a Chlorox solution (0.5%) for 15 minutes after which time the seeds are rinsed with sterile distilled water. The immature cotyledons are then excised by first cutting away the portion of the seed that contains the embryo axis. The cotyledons are then removed from the seed coat by gently pushing the distal end of the seed with the blunt end of the scalpel blade. The cotyledons are then placed (flat side up) in SB1 initiation medium (MS salts, B5 vitamins, 20 mg/L 2,4-D, 31.5 g/l sucrose, 8 g/L TC Agar, pH 5.8). The Petri plates are incubated in the light (16 hr day; 75-80 μE at 26° C. After 4 weeks of incubation the cotyledons are transferred to fresh SB1 medium. After an additional two weeks, globular stage somatic embryos that exhibit proliferative areas are excised and transferred to FN Lite liquid medium (Samoylov, V. M., D. M. Tucker, and W. A. Parrott (1998) Soybean [Glycine max (L.) Merrill] embryogenic cultures: the role of sucrose and total nitrogen content on proliferation. In Vitro Cell Dev. Biol. —Plant 34:8-13). About 10 to 12 small clusters of somatic embryos are placed in 250 ml flasks containing 35 ml of SB172 medium. The soybean embryogenic suspension cultures are maintained in 35 mL liquid media on a rotary shaker, 150 rpm, at 26° C. with florescent lights (20 on a 16:8 hour day/night schedule. Cultures are sub-cultured every two weeks by inoculating approximately 35 mg of tissue into 35 mL of liquid medium.

Soybean embryogenic suspension cultures may then be transformed using particle gun bombardment (Klein et al. (1987) Nature (London) 327:70, U.S. Pat. No. 4,945,050). A BioRad Biolistic™ PDS1000/HE instrument may be used for these transformations. A selectable marker gene which is used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens.

To 50 μL of a 60 mg/mL 1 μm gold particle suspension is added (in order): 5

What is claimed is:

1. A method for increasing transformation efficiency comprising:
(a) introducing into a responsive plant cell under conditions sufficient to increase transformation efficiency a construct comprising a polynucleotide encoding a leafy cotyldon1 (LEC1) variant operably linked to a promoter functional in plant cells to yield transformed plant cells, and wherein the LEC1 variant polynucleotide encoding the LEC1 variant protein is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8).

2. The method of claim 1, wherein the plant cell is transformed with at least one LEC1 variant polynucleotide.

3. The method of claim 2, wherein plant cell is in contact with medium that retards growth of somatic embryo growth in non-transformed plants.

4. The method of claim 2, wherein transformation is conducted with reduced levels of auxin or no auxin.

5. The method of claim 2, wherein the at least one LEC1 variant polynucleotide is operably linked to a promoter driving expression in a plant cell.

6. The method of claim 1, wherein the plant cell is a recalcitrant cell.

7. The method of claim 1, wherein the plant cell is an inbred cell.

8. A method for enhancing tissue culture response in a plant cell comprising introducing into the plant cell a LEC1 variant polynucleotide selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8); and
growing the plant cell to enhance the tissue culture response.

9. The method of claim 8, wherein the plant cell is transformed with at least one LEC1 variant polynucleotide.

10. The method of claim 8, wherein at least one LEC1 variant polynucleotide is operably linked to a promoter driving expression in the plant cell.

11. The method of claim 8, wherein the plant cell is a recalcitrant cell.

12. The method of claim 8, wherein the plant cell is an inbred plant cell.

13. The method of claim 8, wherein LEC1 variant polynucleotide is transiently introduced.

14. The method of claim 8, wherein the plant cell is stably transformed with the LEC1 variant polynucleotide.

15. A method for inducing somatic embryogenesis in a plant cell comprising introducing into a plant cell at least one LEC1 variant polynucleotide that encodes a LEC1 variant polypeptide that induces somatic embryogenesis in said plant cell, wherein the plant cell is grown to produce a transformed embryo and wherein said LEC1 variant polynucleotide is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8).

16. The method of claim 15, wherein the plant cell is transformed with at least one LEC1 variant polynucleotide.

17. The method of claim 15, wherein at least one LEC1 variant polynucleotide is operably linked to a promoter driving expression in the plant cell.

18. The method of claim 15, further comprising growing the transformed embryo under plant growing conditions to produce a regenerated plant.

19. A transgenic plant produced by the method of claim 18.

20. The method of claim 15, wherein the plant cell is from a monocot plant.

21. The method of claim 15, wherein the plant cell is from a dicot plant.

22. The method of claim 15, wherein the plant cell is from corn, soybean, sorghum, wheat, rice, alfalfa, sunflower, canola or cotton.

23. A method for positive selection of a transformed cell comprising transforming a plant cell with a LEC1 variant polynucleotide operably linked to a promoter capable of directing expression in the plant cell, wherein said LEC1 variant polynucleotide is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8); and
growing the transformed plant cell to provide a positive selection means.

24. The method of claim 23, further comprising altering media components to favor the growth of transformed plant cells.

25. The method of claim 24, wherein the media components are altered to reduce somatic embryogenesis in non-transformed cells.

26. The method of claim 23, wherein the plant cell is transformed with at least one LEC1 variant polynucleotide.

27. The method of claim 26, wherein at least one LEC1 variant polynucleotide is operably linked to a promoter driving expression in a plant.

28. The method of claim 24, wherein the LEC1 variant polynucleotide is subsequently excised.

29. The method of claim 27 wherein the LEC1 variant polynucleotide is flanked by FRT sequences to allow FLP mediated excision of the LEC1

30. A method for inducing apomixis in a plant cell comprising introducing a LEC1 variant polynucleotide into a cell of a plant seed and growing the cell of the plant seed to produce a somatic embryo, wherein said LEC1 variant polynucleotide is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8).

31. The method of claim 30, wherein the plant cell is transformed with at least one LEC1 variant polynucleotide.

32. The method of claim 31, wherein the at least one LEC1 variant polynucleotide is operably linked to a promoter driving expression in a plant cell.

33. The method of claim 32, wherein the promoter is an inducible promoter.

34. The method of claim 30, wherein the plant cell is a recalcitrant cell.

35. The method of claim 30, wherein the plant cell is an inbred plant cell.

36. The method of claim 30, wherein the LEC1 variant polynucleotide is transiently introduced.

37. The method of claim 30, wherein the at least one LEC1 variant polynucleotide is expressed in integument or nucellus tissue.

38. The method of claim 30, further comprising growing the transformed somatic embryo under plant growing conditions to produce a regenerated plant.

39. A plant produced by the method of claim 30.

40. The method of claim 39, wherein the plant cell is from a monocot plant or a dicot plant.

41. A method for increasing recovery of regenerated plants comprising introducing into a plant cell a LEC1 variant polynucleotide and growing the plant cell to produce a regenerated plant, wherein said LEC1 variant polynucleotide is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8).

42. The method of claim 41, wherein the plant cell is transformed with at least one LEC1 variant polynucleotide.

43. The method of claim 42, wherein the at least one LEC1 variant polynucleotide is operably linked to a promoter driving expression in a plant cell.

44. The method of claim 41, wherein the plant cell is a recalcitrant cell.

45. The method of claim 41, wherein the plant cell is an inbred plant cell.

46. A method for increasing callus growth rate comprising:
(a) introducing into a responsive plant cell under conditions sufficient for callus to grow a construct comprising a polynucleotide encoding a leafy cotyldon1 (LEC1) variant operably linked to a promoter functional in plant cells to yield transformed plant cells, and wherein the LEC1 variant polynucleotide encoding the LEC1 variant protein is selected from the group consisting of:
(1) an isolated polynucleotide encoding a LEC1 variant polypeptide comprising a B domain of a LEC1 protein and further comprising an A domain and a C domain, wherein at least the A domain or C domain is from a second LEC1 protein and the B domain is from a first LEC1 protein, and wherein the B domain has less than 80% identity to an Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(2) an isolated polynucleotide encoding a LEC1 variant polypeptide comprising an A domain, a B domain, and a C domain, wherein the B domain is mutated so that it has less than 80% identity to the Arabidopsis LEC1 B domain of SEQ ID NO:1 or wherein the B domain comprises an amino acid sequence of Met Pro Ile Ala Asn Val Ile (SEQ ID NO:14) and wherein the sequence has at least one mutation;
(3) an isolated polynucleotide that encodes the polypeptide of SEQ ID NO:5, 7, 9, 11 or 13;
(4) an isolated polynucleotide comprising the sequence set forth in SEQ ID NO:4, 6, 8, 10 or 12; and
(5) an isolated polynucleotide comprising at least 30 nucleotides in length which hybridizes under stringent conditions to a polynucleotide of (a), (b), (c), or (d) wherein the conditions include hybridization in 40 to 45% formamide, 1 M NaCl, 1% SDS at 37° C. and a wash in 0.5× to 1×SSC at 55 to 60° C.; and
(6) an isolated polynucleotide having at least 85%, 90%, or 95% sequence identity to SEQ ID NO:4, 6, 8, 10 or 12, wherein the % sequence identity is based on the entire encoding region and is determined by BLAST 2.0 under default parameters;
(7) a polynucleotide encoding a polypeptide that is at least 85%, 90%, or 95% identical to a polypeptide comprising the sequence set forth in SEQ ID NO: 5, 7, 9, 11 or 13 wherein the encoded polypeptide has Lec1 activity;
(8) an isolated polynucleotide degenerate from any of (1) to (7) as a result of the genetic code; and
(9) an isolated polynucleotide complementary to a polynucleotide of any one of (1) to (8).


SEQUENCE LISTING (29 sequences)

SEQ ID NO: 1 - protein, 90 aa, Arabidopsis thaliana
REQDQYMPIANVIRIMRKTLPSHAKISDDAKETIQECVSEYISFVTGEANERCQREQRKTITAEDILWAMSKLGFDNYVDPLTVFINRYR

SEQ ID NO: 2 - DNA, 270 bp, Zea mays
  CDS (1)..(270)
cgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgc

SEQ ID NO: 3 - protein, 90 aa, Zea mays
REQDRLMPIANVIRIMRRVLPAHAKISDDAKETIQECVSEYISFITGEANERCQREQRKTITAEDVLWAMSRLGFDDYVEPLGAYLHRYR

SEQ ID NO: 4 - DNA, 837 bp, artificial sequence
  Description of artificial sequence Maize LEC1 with mutated B domain.
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtggcccgcatcatgaagcgcgtggtgccggcgcacgcccgcgtgtcggacgacgccaaggagacgctgcaggagtgcgtgtcggagtacatcagcttcatcacgggggaggccaacgagcggtgccgccaggagcagcgcaagaccctgaccgccgaggacatcctgttcgccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 5 - protein, 278 aa, artificial sequence
  Synthetic Construct
MDSSSFLPAAGAENGSAAGGANNGGAAQQHAAPAIREQDRLMPIANVARIMKRVVPAHARVSDDAKETLQECVSEYISFITGEANERCRQEQRKTLTAEDILFAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 6 - DNA, 837 bp, Artificial sequence
  Description of artificial sequence Maize LEC1 with mutated B domain.
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtggcccgcctgatgaagcgcgtgatcccggcgcacgcccgcgtggccgacgacgccaaggagacgctgcaggagtgcgtgtcggagttcatcagcttcatcacgagcgaggccagcgagcggtgccgccaggagcagcgcaagaccatcaccgccgaggacctcctgtgggccctgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 7 - protein, 278 aa, Artificial sequence
  Synthetic Construct
MDSSSFLPAAGAENGSAAGGANNGGAAQQHAAPAIREQDRLMPIANVARLMKRVIPAHARVADDAKETLQECVSEFISFITSEASERCRQEQRKTITAEDLLWALSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 8 - DNA, 837 bp, Artificial sequence
  Description of artificial sequence Maize LEC1 with mutated B domain.
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtggcccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 9 - protein, 278 aa, Artificial sequence
  Synthetic Construct
MDSSSFLPAAGAENGSAAGGANNGGAAQQHAAPAIREQDRLMPIANVARIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 10 - DNA, 837 bp, Artificial sequence
  Description of artificial sequence Maize LEC1 with mutated B domain.
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacatcatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 11 - protein, 278 aa, Artificial sequence
  Synthetic Construct
MDSSSFLPAAGAENGSAAGGANNGGAAQQHAAPAIREQDRLMPIANIIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 12 - DNA, 837 bp, Artificial sequence
  Description of artificial sequence Maize LEC1 with wheat B domain.
atggactccagcagcttcctccctgccgccggcgcggagaatggctcggcggcgggcggcgccaacaatggcggcgctgctcagcagcatgcggcgccggcgatccgggagcaggaccggctgatgccgatcgcgaacgtgatccgcatcatgcgccgtgcgctccctgcccacgccaagatctccgacgacgccaaggaggcgattcaggaatgcgtgtccgagttcatcagcttcgtcaccggcgaggccaacgaacggtgccgcatgcagcaccgcaagaccgtcaacgccgaagacatcgtgtgggccctaaaccgcctcggcttcgacgactacgtcgtgcccctcagcgtcttcctgcaccgcatgcgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 13 - protein, 278 aa, Artificial sequence
  Synthetic Construct
MDSSSFLPAAGAENGSAAGGANNGGAAQQHAAPAIREQDRLMPIANVIRIMRRALPAHAKISDDAKEAIQECVSEFISFVTGEANERCRMQHRKTVNAEDIVWALNRLGFDDYVVPLSVFLHRMREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 14 - protein, 7 aa, Zea mays
MPIANVI

SEQ ID NO: 15 - protein, 208 aa, Arabidopsis thaliana
MTSSVIVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 16 - DNA, 837 bp, Zea mays
  CDS (1)..(834)
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaagtag

SEQ ID NO: 17 - protein, 278 aa, Zea mays
MDSSSFLPAAGAENGSAAGGANNGGAAQQHAAPAIREQDRLMPIANVIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 18 - DNA, 270 bp, Triticum aestivum
  CDS (1)..(267)
cgggagcaggaccggctgatgccgatcgcgaacgtgatccgcatcatgcgccgtgcgctccctgcccacgccaagatctccgacgacgccaaggaggcgattcaggaatgcgtgtccgagttcatcagcttcgtcaccggcgaggccaacgaacggtgccgcatgcagcaccgcaagaccgtcaacgccgaagacatcgtgtgggccctaaaccgcctcggcttcgacgactacgtcgtgcccctcagcgtcttcctgcaccgcatgcgc

SEQ ID NO: 19 - protein, 89 aa, Triticum aestivum
REQDRLMPIANVIRIMRRALPAHAKISDDAKEAIQECVSEFISFVTGEANERCRMQHRKTVNAEDIVWALNRLGFDDYVVPLSVFLHRM

SEQ ID NO: 20 - DNA, 843 bp, Triticum aestivum
  CDS (1)..(840)
atggagaacgacggcgtccccaacggaccagcggcgccggcacctacccaggggacgccggtggtgcgggagcaggaccggctgatgccgatcgcgaacgtgatccgcatcatgcgccgtgcgctccctgcccacgccaagatctccgacgacgccaaggaggcgattcaggaatgcgtgtccgagttcatcagcttcgtcaccggcgaggccaacgaacggtgccgcatgcagcaccgcaagaccgtcaacgccgaagacatcgtgtgggccctaaaccgcctcggcttcgacgactacgtcgtgcccctcagcgtcttcctgcaccgcatgcgcgaccccgaggcggggacaggtggtgccgctgcaggcgacagccgcgccgtgacgagtgcgcctccccgcgcggccccgcccgtgatccacgccgtgccgctgcaggctcagcgcccgatgtacgcgcccccggctccgttgcaggttgagaatcagatgcagcggcctgtgtacgctcccccggctccggtgcaggttcagatgcagcggggcatctatgggccccgggctccagtgcacgggtacgccgtcggaatggcgcccgtgcgggccaacgtcggcgggcagtaccaggtgttcggcggagagggtgtcatggcccagcaatactacgggtacgggtacgaggaaggagcgtacggcgcaggtagcagcaacggaggagccgccattggcgacgaggagagctcgtccaacggcgtgccggcaccgggggagggcatgggggagccagagccagagccagcagcagaagaatcgcatgacaagcccgtccaatctggctag

SEQ ID NO: 21 - protein, 280 aa, Triticum aestivum
MENDGVPNGPAAPAPTQGTPVVREQDRLMPIANVIRIMRRALPAHAKISDDAKEAIQECVSEFISFVTGEANERCRMQHRKTVNAEDIVWALNRLGFDDYVVPLSVFLHRMRDPEAGTGGAAAGDSRAVTSAPPRAAPPVIHAVPLQAQRPMYAPPAPLQVENQMQRPVYAPPAPVQVQMQRGIYGPRAPVHGYAVGMAPVRANVGGQYQVFGGEGVMAQQYYGYGYEEGAYGAGSSNGGAAIGDEESSSNGVPAPGEGMGEPEPEPAAEESHDKPVQSG

SEQ ID NO: 22 - DNA, 270 bp, Artificial sequence
  Description of artificial sequence Maize LEC1 mutated B domain.
cgcgagcaggaccggctgatgccgatcgcgaacgtggcccgcatcatgaagcgcgtggtgccggcgcacgcccgcgtgtcggacgacgccaaggagacgctgcaggagtgcgtgtcggagtacatcagcttcatcacgggggaggccaacgagcggtgccgccaggagcagcgcaagaccctgaccgccgaggacatcctgttcgccatgagccgcctcggcttcgacgactacgtcgagccgctcggcgcctacctccaccgctaccgc

SEQ ID NO: 23 - protein, 89 aa, Artificial sequence
  Synthetic Construct
REQDRLMPIANVARIMKRVVPAHARVSDDAKETLQECVSEYISFITGEANERCRQEQRKTLTAEDILFAMSRLGFDDYVEPLGAYLHRY

SEQ ID NO: 24 - DNA, 270 bp, Artificial sequence
  Description of artificial sequence Maize LEC1 mutated B domain.
cgcgagcaggaccggctgatgccgatcgcgaacgtggcccgcctgatgaagcgcgtgatcccggcgcacgcccgcgtggccgacgacgccaaggagacgctgcaggagtgcgtgtcggagttcatcagcttcatcacgagcgaggccagcgagcggtgccgccaggagcagcgcaagaccatcaccgccgaggacctcctgtgggccctgagccgcctcggcttcgacgactacgtcgagccgctcggcgcctacctccaccgctaccgc

SEQ ID NO: 25 - protein, 89 aa, Artificial sequence
  Synthetic Construct
REQDRLMPIANVARLMKRVIPAHARVADDAKETLQECVSEFISFITSEASERCRQEQRKTITAEDLLWALSRLGFDDYVEPLGAYLHRY

SEQ ID NO: 26 - DNA, 270 bp, Artificial sequence
  Description of artificial sequence Maize LEC1 mutated B domain.
cgcgagcaggaccggctgatgccgatcgcgaacgtggcccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgc

SEQ ID NO: 27 - protein, 89 aa, Artificial sequence
  Synthetic Construct
REQDRLMPIANVARIMRRVLPAHAKISDDAKETIQECVSEYISFITGEANERCQREQRKTITAEDVLWAMSRLGFDDYVEPLGAYLHRY

SEQ ID NO: 28 - DNA, 270 bp, Artificial sequence
  Description of artificial sequence Maize LEC1 mutated B domain.
cgcgagcaggaccggctgatgccgatcgcgaacatcatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgc

SEQ ID NO: 29 - protein, 89 aa, Artificial sequence
  Synthetic Construct
REQDRLMPIANIIRIMRRVLPAHAKISDDAKETIQECVSEYISFITGEANERCQREQRKTITAEDVLWAMSRLGFDDYVEPLGAYLHRY